(19) United States
(12) Patent Application Publication
(10) Pub. No.: US 2009/0104281 A1
(43) Pub. Date: Apr. 23, 2009

(54) Compositions Having a High Antiviral and Antibacterial Efficacy

(75) Inventors: Timothy J. Taylor, Phoenix, AZ (US); Harry E. Towner, Scottsdale, AZ (US); Janice L. Fuls, Fountain Hills, AZ (US); Bruce R. Cox, Scottsdale, AZ (US); George E. Fischler, Scottsdale, AZ (US); Priscilla S. Fox, Phoenix, AZ (US); Nancy D. Rodgers, Chandler, AZ (US); James Dalton, Scottsdale, AZ (US)

Correspondence Address: MARSHALL, GERSTEIN & BORUN LLP (DIAL), 233 S. WACKER DRIVE, 6300 SEARS TOWER, CHICAGO, IL, 60606, US

(73) Assignee: THE DIAL CORPORATION, Scottsdale, AZ (US)

(21) Appl. No.: 11/791,301

(22) PCT Filed: Dec. 5, 2005
(86) PCT No.: PCT/US05/43794

§ 371 Date: Aug. 25, 2008

Related U.S. Application Data
(60) Provisional application No. 60/634,483, filed on Dec. 9, 2004.

(51) Int. Cl. A61K 33/42; A61K 33/06; A61K 33/24; A61K 33/00; A61K 31/19; A61K 31/66

(52) U.S. Cl. 424/605; 424/682; 424/617; 514/557; 514/574; 514/75; 424/601; 424/722

(57) ABSTRACT

A method of providing a rapid, broad spectrum bacterial control, and a rapid persistent antiviral control on a surface, and particularly a mammalian skin surface, is disclosed. In the method, a compound or composition capable of lowering skin pH to less than about 4 is applied to the skin, and preferably is allowed to remain on the skin.

### CROSS REFERENCE TO RELATED APPLICATIONS

This application claims the benefit of U.S. provisional patent application No. 60/634,483, filed Dec. 9, 2004.

### FIELD OF THE INVENTION

The present invention relates to a method of providing a rapid and a persistent control of viruses, and a rapid, broad-spectrum control of bacteria, on a surface, and particularly on mammalian skin. More particularly, the present invention relates to a method of controlling viruses and bacteria on mammalian skin by applying a compound or a composition to the skin that is capable of providing a skin pH of less than about 4, for a period of about four or more hours, without irritating the skin. The compound typically is (a) an organic acid, (b) an inorganic acid, (c) an inorganic salt, (d) an aluminum, zirconium, or aluminum-zirconium complex, or (e) mixtures thereof, capable of sufficiently lowering mammalian skin pH to control viruses and bacteria. The surface optionally can be contacted with one or both of a disinfecting alcohol and an antimicrobial agent to assist in bacterial and viral control. The method controls Gram positive and Gram negative bacterial populations, and viral populations, within one minute, and provides a persistent antiviral control for about four hours or more.

### BACKGROUND OF THE INVENTION

Human health is impacted by a variety of microbes encountered on a daily basis. In particular, contact with various microbes in the environment can lead to an illness, possibly severe, in mammals. For example, microbial contamination can lead to a variety of illnesses, including, but not limited to, food poisoning, a streptococcal infection, anthrax (cutaneous), athlete's foot, cold sores, conjunctivitis (“pink eye”), coxsackievirus (hand-foot-mouth disease), croup, diphtheria (cutaneous), ebolic hemorrhagic fever, and impetigo.

It is known that washing body parts (e.g., hand washing) and hard surfaces (e.g., countertops and sinks) can significantly decrease the population of microorganisms, including pathogens. Therefore, cleaning skin, and other animate and inanimate surfaces, to reduce microbial populations is a first defense in removing such pathogens from these surfaces, and thereby minimizing the risk of infection.

Viruses are one category of pathogens that are of primary concern. Viral infections are among the greatest causes of human morbidity, with an estimated 60% or more of all episodes of human illness in developed countries resulting from a viral infection. In addition, viruses infect virtually every organism in nature, with high virus infection rates occurring among all mammals, including humans, pets, livestock, and zoo specimens.

Viruses exhibit an extensive diversity in structure and lifecycle. A detailed description of virus families, their structures, life cycles, and modes of viral infection is discussed in Fundamental Virology, 4th Ed., Eds. Knipe & Howley, Lippincott Williams & Wilkins, Philadelphia, Pa., 2001.

Simply stated, virus particles are intrinsic obligate parasites, and have evolved to transfer genetic material between cells and encode sufficient information to ensure their own propagation. In a most basic form, a virus consists of a small segment of nucleic acid encased in a simple protein shell. The broadest distinction between viruses is the enveloped and nonenveloped viruses, i.e., those that do or do not contain, respectively, a lipid-bilayer membrane.

Viruses propagate only within living cells. The principal obstacle encountered by a virus is gaining entry into the cell, which is protected by a cell membrane of thickness comparable to the size of the virus. In order to penetrate a cell, a virus first must become attached to the cell surface. Much of the specificity of a virus for a certain type of cell lies in its ability to attach to the surface of that specific cell. Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell. Therefore, in order to control a viral infection, it is important to rapidly kill a virus that contacts the skin, and ideally to provide a persistent antiviral activity on the skin, or a hard surface, in order to control viral infections.

For example, rhinoviruses, influenza viruses, and adenoviruses are known to cause respiratory infections. Rhinoviruses are members of the picornavirus family, which is a family of “naked viruses” that lack an outer envelope. The human rhinoviruses are so termed because of their special adaptation to the nasopharyngeal region, and are the most important etiological agents of the common cold in adults and children. Officially, there are 102 rhinovirus serotypes. Most of the picornaviruses isolated from the human respiratory system are acid labile, and this liability has become a defining characteristic of rhinoviruses.

Rhinovirus infections are spread from person to person by direct contact with virus-contaminated respiratory secretions. Typically, this contact is in the form of physical contact with a contaminated surface, rather than via inhalation of airborne viral particles.

Rhinovirus can survive on environmental surfaces for hours after initial contamination. Rhinovirus infection is readily transmitted by finger-to-finger contact, and by contaminated environmental surface-to-finger, contact, when the newly contaminated finger then rubs an eye or touches the nasal mucosa. Therefore, virus contamination of skin and environmental surfaces should be minimized to reduce the risk of transmitting the infection to the general population.

Several gastrointestinal infections also are caused by viruses. For example, Norwalk virus causes nausea, vomiting (sometimes accompanied by diarrhea), and stomach cramps. This infection typically is spread from person to person by direct contact. Acute hepatitis A viral infection similarly can be spread by direct contact between one infected person and a nonimmune individual by hand-to-hand, hand-to-mouth, or aerosol droplet transfer, or by indirect contact when an uninfected individual comes into contact with a hepatitis A virus-contaminated solid object. Numerous other viral infections are spread similarly. The risk of transmitting such viral infections can be reduced significantly by inactivating or removing viruses from the hands and other environmental surfaces.

Common household phenol/alcohol disinfectants are effective in disinfecting contaminated environmental surfaces, but lack persistent virucidal activity. Hand washing is highly effective in disinfecting contaminated fingers, but again suffers from a lack of persistent activity. These shortcomings illustrate the need for improved virucidal compositions having a persistent activity against viruses, such as rhinoviruses.

Antimicrobial personal care compositions are known in the art. In particular, antibacterial cleansing compositions, which typically are used to cleanse the skin and to destroy bacteria present on the skin, especially the hands, arms, and face of the user, are well-known commercial products.

Antibacterial compositions are used, for example, in the health care industry, food service industry, meat processing industry, and in the private sector by individual consumers. The widespread use of antibacterial compositions indicates the importance consumers place on controlling bacteria populations on skin. The paradigm for antibacterial compositions is to provide a substantial and broad spectrum reduction in bacterial populations quickly and without adverse side effects associated with toxicity and skin irritation. Such antibacterial compositions are disclosed in U.S. Pat. Nos. 6,107,261 and 6,136,771, each incorporated herein by reference.

One class of antibacterial personal care compositions is the hand sanitizer gels. This class of compositions is used primarily by medical personnel to disinfect the hands and fingers. A hand sanitizer gel is applied to, and rubbed into, the hands and fingers, and the composition is allowed to evaporate from the skin.

Hand sanitizer gels contain a high percentage of an alcohol, like ethanol. At the high percent of alcohol present in the gel, the alcohol itself acts as a disinfectant. In addition, the alcohol quickly evaporates to obviate wiping or rinsing skin treated with the sanitizer gel. Hand sanitizer gels containing a high percentage of an alcohol, i.e., about 40% or greater by weight of the composition, do not provide a persistent bacterial kill.

Antibacterial cleansing compositions typically contain an active antibacterial agent, a surfactant, and various other ingredients, for example, dyes, fragrances, pH adjusters, skin conditioners, and the like, in an aqueous and/or alcoholic carrier. Several different classes of antibacterial agents have been used in antibacterial cleansing compositions. Examples of antibacterial agents include bisguanidines (e.g., chlorhexidine gluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxydiphenylether). Antimicrobial compositions based on such antibacterial agents exhibit a wide range of antibacterial activity, ranging from low to high, depending on the microorganism to be controlled and the particular antibacterial composition.

Most commercial antibacterial compositions generally offer a low to moderate antibacterial activity, and no reported antiviral activity. Antibacterial activity is assessed against a broad spectrum of microorganisms, including both Gram positive and Gram negative microorganisms. The log reduction, or alternatively the percent reduction, in bacterial populations provided by the antibacterial composition correlates to antibacterial activity. A 1-3 log reduction is preferred, a log reduction of 3-5 is most preferred, whereas a log reduction of less than 1 is least preferred, for a particular contact time, generally ranging from 15 seconds to 5 minutes. Thus, a highly preferred antibacterial composition exhibits a 3-5 log reduction against a broad spectrum of microorganisms in a short contact time.

Virus control poses a more difficult problem, however. By sufficiently reducing bacterial populations, the risk of bacterial infection is reduced to acceptable levels. Therefore, a rapid antibacterial kill is desired. With respect to viruses, however, not only is a rapid kill desired, but a persistent antiviral activity also is required. This difference is because merely reducing a viral population is insufficient to reduce infection. In theory, a single virus can cause infection. Therefore, an essentially total, and persistent, antiviral activity is required, or at least desired, for an effective antiviral cleansing composition.

WO 98/01110 discloses compositions comprising triclosan, surfactants, solvents, chelating agents, thickeners, buffering agents, and water. WO 98/01110 is directed to reducing skin irritation by employing a reduced amount of surfactant.

U.S. Pat. No. 5,635,462 discloses compositions comprising PCMX and selected surfactants. The compositions disclosed therein are devoid of anionic surfactants and nonionic surfactants.

EP 0 505 935 discloses compositions containing PCMX in combination with nonionic and anionic surfactants, particularly nonionic block copolymer surfactants.

WO 95/32705 discloses a mild surfactant combination that can be combined with antibacterial compounds, like triclosan.

WO 95/09605 discloses antibacterial compositions containing anionic surfactants and alkylpolyglycoside surfactants.

WO 98/55096 discloses antimicrobial wipes having a porous sheet impregnated with an antibacterial composition containing an active antimicrobial agent, an anionic surfactant, an acid, and water, wherein the composition has a pH of about 3.0 to about 6.0.

U.S. Pat. No. 6,110,908 discloses a topical antiseptic containing a C₂₋₃ alcohol, a free fatty acid, and zinc pyrithione.

N. A. Allawala et al., J. Amer. Pharm. Assoc. —Sci. Ed., Vol. XLII, no. 5, pp. 267-275 (1953) discusses the antibacterial activity of active antibacterial agents in combination with surfactants.

A. G. Mitchell, J. Pharm. Pharmacol., Vol. 16, pp. 533-537 (1964) discloses compositions containing PCMX and a nonionic surfactant that exhibit antibacterial activity.

With respect to hand sanitizer gels, U.S. Pat. No. 5,776,430 discloses a topical antimicrobial cleaner containing chlorhexidine and an alcohol. The compositions contain about 50% to 60%, by weight denatured alcohol and about 0.65% to 0.85%, by weight chlorhexidine. The composition is applied to the skin, scrubbed into the skin, then rinsed from the skin.

European Patent Application 0 604 848 discloses a gel-type hand disinfectant containing an antimicrobial agent, 40% to 90% by weight of an alcohol, and a polymer and a thickening agent in a combined weight of not more than 3% by weight. The gel is rubbed into the hands and allowed to evaporate to provide disinfected hands. The disclosed compositions often do not provide immediate sanitization and do not provide persistent antimicrobial efficacy.

In general, hand sanitizer gels typically contain: (a) at least 60% by weight ethanol or a combination of lower alcohols, such as ethanol and isopropanol, (b) water, (c) a gelling polymer, such as a crosslinked polyacrylate material, and (d) other ingredients, such as skin conditioners, fragrances, and the like. Hand sanitizer gels are used by consumers to effectively sanitize the hands, without, or after; washing with soap and water, by rubbing the hand sanitizer gel on the surface of the hands. Current commercial hand sanitizer gels rely on high levels of alcohol for disinfection and evaporation, and thus suffer from disadvantages. Specifically, because of the volatility of ethanol, the primary active disinfectant does not remain on the skin after use, thus failing to provide a persistent antimicrobial effect.

At alcohol concentrations below 60%, ethanol is not recognized as an antiseptic. Thus, in compositions containing less than 60% alcohol, an additional antimicrobial compound typically is present to provide antimicrobial activity. Prior disclosures, however, have not addressed the issue of which composition ingredient in such an antimicrobial composition provides microbe control. Therefore, for formulations containing a reduced alcohol concentration, the selection of an antimicrobial agent that provides both a rapid antimicrobial effect and a persistent antimicrobial benefit is difficult.

U.S. Pat. Nos. 6,107,261 and 6,136,771 disclose highly effective antibacterial compositions. These patents disclose compositions that solve the problem of controlling bacteria on skin and hard surfaces, but are silent with respect to controlling viruses.

U.S. Pat. Nos. 5,968,539; 6,106,851; and 6,113,933 disclose antibacterial compositions having a pH of about 3 to about 6. The compositions contain an antibacterial agent, an anionic surfactant, and a proton donor.

A composition containing a quaternary ammonium compound and a selected anionic surfactant has been disclosed as being effective in some applications (e.g., U.S. Pat. No. 5,798,329), but no reference disclosing such a combination for use in personal care compositions has been found.

Patents and published applications disclosing germicidal compositions containing a quaternary ammonium antibacterial agent include U.S. Pat. Nos. 5,798,329 and 5,929,016; WO 97/15647; and EP 0 651 048, directed to antibacterial laundry detergents and antibacterial hard surface cleaners.

Antiviral compositions that inactivate or destroy pathogenic viruses, including rhinovirus, rotavirus, influenza virus, parainfluenza virus, respiratory syncytial virus, and Norwalk virus, also are known. For example, U.S. Pat. No. 4,767,788 discloses the use of glutaric acid to inactivate or destroy viruses, including rhinovirus. U.S. Pat. No. 4,975,217 discloses compositions containing an organic acid and an anionic surfactant, for formulation as a soap or lotion, to control viruses. U.S. Patent Publication 2002/0098159 discloses the use of a proton donating agent and a surfactant, including an antibacterial surfactant, to effect antiviral and antibacterial properties.

U.S. Pat. No. 6,034,133 discloses a virucidal hand lotion containing malic acid, citric acid, and a C₁₋₆ alcohol. U.S. Pat. No. 6,294,186 discloses combinations of a benzoic acid analog, such as salicyclic acid, and selected metal salts as being effective against viruses, including rhinovirus. U.S. Pat. No. 6,436,885 discloses a combination of known antibacterial agents with 2-pyrrolidone-5-carboxylic acid, at a pH of 2 to 5.5, to provide antibacterial and antiviral properties.

Organic acids in personal washing compositions also have been disclosed. For example, WO 97/46218 and WO 96/06152 disclose the use of organic acids or salts, hydrotropes, triclosan, and hydric solvents in a surfactant base for antimicrobial cleansing compositions. These publications are silent with respect to antiviral properties.

Hayden et al., Antimicrobial Agents and Chemotherapy, 26:928-929 (1984), discloses interrupting the hand-to-hand transmission of rhinovirus colds through the use of a hand lotion having residual virucidal activity. The hand lotions, containing 2% glutaric acid, were more effective than a placebo in inactivating certain types of rhinovirus. However, the publication discloses that the glutaric acid-containing lotions were not effective against a wide spectrum of rhinovirus serotypes.

A virucidal tissue designed for use by persons infected with the common cold, and including citric acid, malic acid, and sodium lauryl sulfate, is known. Hayden et al., Journal of Infectious Diseases, 152:493-497 (1985), however, reported that use of paper tissues, either treated with virus-killing substances or untreated, can interrupt the hand-to-hand transmission of viruses. Hence, no distinct advantage in preventing the spread of rhinovirus colds can be attributed to the compositions incorporated into the virucidal tissues.

U.S. Pat. No. 4,503,070 discloses a method of treating a common cold by the topical application of zinc gluconate to the oral mucosa. The method reduces the duration of the cold by alleviating common cold symptoms. U.S. Pat. No. 5,409,905 also discloses a method of treating a common cold by applying a solid composition containing zinc ions to the oral and oropharyngeal membranes of a human. U.S. Pat. No. 5,622,724 discloses a treatment for the common cold comprising administering a spray comprising a solution of a substantially unchelated ionic zinc compound to the nostrils and respiratory tract of a patient in need. U.S. Pat. No. 6,673,835 discloses a method and composition for delivering a low, but effective, amount of a zinc-containing active ingredient into the blood via application to the nasal cavity.

An efficacious method of controlling both bacterial and viral populations has been difficult to achieve because of the fundamental differences between a bacteria and a virus. Even more difficult to achieve is a method that provides a persistent antiviral activity. Although a number of antimicrobial products currently exist, taking a variety of product forms (e.g., deodorant soaps, hard surface cleaners, and surgical disinfectants), such antimicrobial products typically incorporate high levels of an alcohol and/or surfactants, which can dry out and irritate skin tissues. Ideally, personal antimicrobial compositions and methods gently cleanse the skin, cause little or no irritation, and do not leave the skin overly dry after frequent use.

Accordingly, a need exists for a method that is highly efficacious in controlling a broad spectrum of microbes, including viruses and Gram positive and Gram negative bacteria on surfaces, and especially on mammalian skin, in a short time period, and wherein the method provides a persistent antiviral activity, and is mild to the skin. Methods providing an improved reduction in virus and bacteria populations are achieved by the present invention, including methods of providing a persistent reduction in virus populations.

### SUMMARY OF THE INVENTION

The present invention is directed to a method that provides a rapid antiviral and antibacterial control, and a persistent antiviral control, on surfaces, and particularly on mammalian skin. The method provides a substantial viral control and a substantial reduction in Gram positive and Gram negative bacteria in less than about one minute.

More particularly, the present invention provides a method of killing a broad spectrum of bacteria, including Gram positive and Gram negative bacteria such as S. aureus, Salmonella choleraesuis, E. coli, and K. pneumoniae, while simultaneously inactivating or destroying viruses harmful to human health, particularly acid-labile viruses, and especially rhinoviruses and other acid-labile picornaviruses.

Accordingly, one aspect of the present invention is to provide a method of controlling viruses and bacteria on mammalian skin comprising contacting the skin with a compound or composition capable of lowering skin pH to less than about 4, without irritating the skin. In some embodiments, the method provides a broad spectrum bacterial control and a persistent viral control for up to about eight hours.

Another aspect of the present invention is to provide a method of controlling bacteria and viruses on a mammalian skin comprising applying a composition containing an organic acid, an inorganic acid, an inorganic salt, an aluminum, zirconium, or aluminum-zirconium complex, or mixtures thereof, to the skin to sufficiently lower skin pH and thereby control bacteria and viruses, without irritating the skin.

Still another aspect of the present invention is to provide a method of controlling bacteria and viruses on mammalian skin, for an extended time, comprising contacting the skin with an aqueous antimicrobial composition containing a compound selected from the group consisting of (a) an organic acid selected from the group consisting of a monocarboxylic acid, a polycarboxylic acid, a polymeric acid having a plurality of carboxylic, phosphate, sulfonate, and/or sulfate moieties, and mixtures thereof; (b) an inorganic acid that is nonirritating to the skin; (c) an inorganic salt comprising a cation having a valence of 2, 3, or 4 and a counterion, (d) an aluminum, zirconium, or aluminum-zirconium complex, and (e) mixtures thereof, wherein the composition is capable of reducing skin pH to less than about 4.

Another aspect of the present invention is to provide a method that achieves a substantial, wide spectrum bacterial control, and persistent viral control, on mammalian skin.

Yet another aspect of the present invention is to provide a method that achieves a log reduction against Gram positive bacteria (i.e., S. aureus) of at least 2 after 30 seconds of contact.

Still another aspect of the present invention is to provide a method that achieves a log reduction against Gram negative bacteria (i.e., E. coli) of at least 2.5 after 30 seconds of contact.

Another aspect of the present invention is to provide a method that achieves a log reduction against acid-labile viruses, including rhinovirus serotypes, such as Rhinovirus 1a, Rhinovirus 14, Rhinovirus 2, and Rhinovirus 4, of at least 4 on mammalian skin after 30 seconds of contact. The antimicrobial composition also provides a log reduction against nonenveloped viruses of at least 3 for at least about five hours, and at least 2 for about six hours, after application with a 30 second contact time. In some embodiments, the antimicrobial composition provides a log reduction against nonenveloped viruses of 2 for up to about eight hours.

Another aspect of the present invention is to provide a method that achieves a persistent antiviral activity, e.g., about four hours or more, after application of a compound or composition to the skin.

Yet another aspect of the present invention is to provide consumer products, for example, a skin cleanser, a body splash, a surgical scrub, a wound care agent, a hand sanitizer gel, a disinfectant, a pet shampoo, a hard surface sanitizer, a lotion, an ointment, a cream, and the like, capable of reducing the pH of a surface, like mammalian skin, to less than about 4 to effect a rapid, broad spectrum, bacterial control and a persistent viral control, without irritating the skin. The consumer product can be a rinse-off product or a leave-on product. Preferably, the product is allowed to remain on the skin to allow the pH lowering components of the product to remain on, and in some cases substantively deposit on, the skin to enhance a persistent antiviral control.

A further aspect of the present invention is to provide a method of quickly controlling a wide spectrum of viruses and the Gram positive and/or Gram negative bacteria populations on animal tissue, including human tissue, by contacting the tissue, like the dermis, with a compound or composition for a sufficient time, for example, about 15 seconds to 5 minutes or longer, to reduce tissue pH to less than about 4 and thereby reduce bacterial and viral populations to a desired level. A further aspect of the present invention is to provide a method that achieves a persistent control of viruses on animal tissue.

Still another aspect of the present invention is to provide a method of preventing virus-mediated diseases and conditions caused by rhinoviruses, picornaviruses, adenoviruses, rotaviruses, and similar pathogenic viruses.

Yet another aspect of the present invention is to provide a method of interrupting transmission of a virus from animate and inanimate surfaces to an animate surface, especially mammalian skin. Especially provided is a method for controlling the transmission of rhinovirus by effectively controlling rhinoviruses present on human skin and continuing to control rhinoviruses for a period of about four hours or more, and up to about eight hours, after application of a suitable compound or composition to the skin.

These and other novel aspects and advantages of the present invention are set forth in the following, nonlimiting detailed description of the preferred embodiments.

### DETAILED DESCRIPTION OF THE PREFERRED EMBODIMENTS

Personal care products incorporating an active antimicrobial agent have been known for many years. Since the introduction of antimicrobial personal care products, many claims have been made that such products provide antimicrobial properties. To be most effective, an antimicrobial composition should provide a high log reduction against a broad-spectrum of organisms in as short a contact time as possible. Ideally, the composition also should inactivate viruses.

As presently formulated, most commercial liquid antibacterial soap compositions provide a poor to marginal time kill efficacy, i.e., rate of killing bacteria. These compositions do not effectively control viruses.

Antimicrobial hand sanitizer compositions typically do not contain a surfactant and rely upon a high concentration of an alcohol to control bacteria. The alcohols evaporate and, therefore, cannot provide a persistent microbial control. The alcohols also can dry and irritate the skin.

Most current products especially lack efficacy against Gram negative bacteria, such as E. coli, which are of particular concern to human health. Compositions do exist, however, that have an exceptionally high broad spectrum antibacterial efficacy, as measured by a rapid kill of bacteria (i.e., time kill), which is to be distinguished from persistent kill. These products also lack a sufficient antiviral activity.

The present method is directed to providing an excellent broad spectrum antibacterial efficacy and a significantly improved antiviral efficacy compared to prior methods and compositions that utilize a high percentage of an alcohol, i.e., 40% or greater, by weight.

The basis of this improved efficacy is the discovery that reducing the pH of a surface, such as mammalian skin, including human skin, provides a rapid, broad spectrum control of bacteria and a rapid and persistent control of viruses.

Although compositions containing an antimicrobial agent, like triclosan, have demonstrated a rapid and effective antibacterial activity against Gram positive and Gram negative bacteria, control of viruses has been inadequate. Virus control on skin and inanimate surfaces is very important in controlling the transmission of numerous diseases.

For example, rhinoviruses are the most significant microorganisms associated with the acute respiratory illness referred to as the “common cold.” Other viruses, such as parainfluenza viruses, respiratory syncytial viruses (RSV), enteroviruses, and coronaviruses, also are known to cause symptoms of the “common cold,” but rhinoviruses are theorized to cause the greatest number of common colds. Rhinoviruses also are among the most difficult of the cold-causing viruses to control, and have an ability to survive on a hard dry surface for more than four days. In addition, most viruses are inactivated upon exposure to a 70% ethanol solution. However, rhinoviruses remain viable upon exposure to ethanol.

Because rhinoviruses are the major known cause of the common cold, it is important that a composition having antiviral activity controls rhinovirus serotypes. Although the molecular biology of rhinoviruses is now understood, finding effective methods for preventing colds caused by rhinoviruses, and for preventing the spread of the virus to noninfected subjects, has been fruitless.

It is known that iodine is an effective antiviral agent, and provides persistent antirhinoviral activity on skin. In experimentally induced and natural cold transmission studies, subjects who used iodine products had significantly fewer colds than placebo users. This indicates that iodine is effective for prolonged periods at blocking the transmission of rhinoviral infections. Thus, the development of products that deliver both immediate and persistent antiviral activity would be effective in reducing the incidence of colds. Likewise, a topically applied composition that exhibits antiviral activity would be effective in preventing and/or treating diseases caused by other acid-labile viruses.

Virucidal means capable of inactivating or destroying a virus. As used herein, the term “persistent antiviral efficacy” or “persistent antiviral activity” means leaving a residue or imparting a condition on animate (e.g., skin) or inanimate surfaces that provides significant antiviral activity for an extended time after application. A method of the present invention provides a persistent antiviral efficacy, i.e., preferably a log reduction of at least 3, and more preferably a log reduction of at least log 4, against pathogenic acid-labile viruses, such as rhinovirus serotypes, within 30 seconds. Antiviral activity is maintained for at least about 0.5 hour, preferably at least about 1 hour, and more preferably at least about two hours, at least about three hours, or at least about four hours after contact with a suitable compound or composition. In some preferred embodiments, antiviral activity is maintained for about six to about eight hours after contact with the compound or composition. The methodology utilized to determine a persistent antiviral efficacy is discussed below.

The method of the present invention, therefore, is highly effective in providing a rapid and broad spectrum control of bacteria, and a rapid and persistent control of viruses. It has been discovered that persistent antiviral benefits can be imparted to mammalian skin by reducing the skin pH to less than about 4, preferably less than about 3.75, and more preferably less than about 3.5, and most preferably less than about 3.25 by any safe and effective means, typically by contacting the skin with a suitable compound or composition.

Compounds and compositions effective at inactivating or otherwise destroying bacteria and viruses are known, but these compositions and methods rely on the pH of the composition and/or the active ingredients of the compositions to effect viral and bacterial control. Surprisingly, it has been discovered that a rapid and broad spectrum bacterial control, and a persistent viral control, can be achieved by reducing a skin surface pH to less than about 4. Thus, the present method provides a safer, milder, and more efficacious approach to the problem of viral and bacterial control than prior methods and compositions.

The method not only is mild to the skin, but also noncorrosive to inanimate surfaces. Thus, an effective method that solves the problem of bacterial and viral control an inanimate surface also is provided.

The present method comprises contacting a surface, and particularly mammalian skin, with a compound or a composition that lowers the pH of the surface to less than about 4, such as down to about 2.5. Thus, present method is highly efficacious in personal care applications (e.g., using lotions, shower gels, soaps, shampoos, and wipes), industrial and hospital applications (e.g., sterilization of instruments; medical devices, and gloves), and household cleaning applications (e.g., hard surfaces, like floors, countertops, tubs, dishes, and softer cloth materials, like clothing). The present method efficaciously and rapidly disinfects surfaces that are infected or contaminated with Gram negative bacteria, Gram positive bacteria, and acid-labile viruses (e.g., rhinoviruses). The present method also provides a persistent antiviral effectiveness.

The present method can be used in vitro and in vivo. In vitro means in or on nonliving things, especially on inanimate objects having hard or soft surfaces located or used where preventing viral transmission is desired, most especially on objects that are touched by human hands. In vivo means in or on animate objects, especially on mammal skin, and particularly on hands.

The present method comprises contacting a surface with a compound or a composition that reduces skin pH to less than about 4, and preferably less than about 3.75, less than about 3.5, less than abut 3.25, less than about 3.0, and down to a pH of about 2.5, and that maintains a low skin pH over a period of up to about four hours, and in some embodiments up to about eight hours. The compound is applied to the skin in an amount of at least 10 micrograms of the compound per square centimeter of skin surface. The method is highly effective in controlling a broad spectrum of bacteria, including Gram positive and Gram negative bacteria, such as S. aureus, Salmonella choleraesuis, E. coli, and K. pneumoniae, as well as simultaneously inactivating or otherwise destroying viruses harmful to human health, especially rhinovirus, for extended periods of time of about four hours or longer.

In particular, the present method comprises contacting a surface in a transient fashion, such as washing and rinsing, or contacting a surface over a longer period, such as by applying a lotion, cream, gel, or other semisolid without rinsing, with a compound or composition capable of reducing the pH of the surface to less than about 4, and more preferably below about 3.75, for a period of time of up to about five hours, in preferred embodiments up to about eight hours, and at least about one-half hour.

As discussed more fully hereafter, compounds capable of lowering the surface pH include, but are not limited to, (a) an organic acid, preferably an acid that is substantive to the surface and having a pKa of about 1 to about 6, more preferably about 2 to about 5.5, most preferably about 2.5 to about 5, wherein pKa is the negative base ten logarithm of the acid dissociation constant of the acid in water at room temperature (25° C.), including organic polymeric acids, preferably capable of forming a substantive film on a skin surface and having a glass transition temperature, Tg, of less than about 25° C., preferably less than about 20° C., and more preferably less than about 15° C.; (b) an inorganic acid that is noncorrosive to the skin and other surfaces; (c) an inorganic salt solution, such as a solution of a salt MX wherein M is a multivalent cation and X is an anion such that MX has a solubility in water of at least 0.1 g/100 ml at 25° C. and the pH of the solution is less than about 6, preferably less than about 5, more preferably less than about 4.5; (d) an aluminum, zirconium, or aluminum-zirconium complex; and (e) mixtures thereof.

The above and other compounds capable of lowering skin pH can be incorporated into consumer-acceptable compositions for an effective and esthetic application to the skin. Such compositions can contain other ingredients, such as additional antimicrobial agents, like a triclosan, a trichlorocarbanilide, a quaternary ammonium antimicrobial agent, a pyrithione salt, and a cosmetic preservative, and similar compounds, in an amount of from 0% to about 5%, by weight of the composition.

The present method exhibits a log reduction against Gram positive bacteria of about 2 after 30 seconds contact. The method also exhibits a log reduction against Gram negative bacteria of about 2.5 after 30 seconds contact. In addition to a rapid control of Gram positive and Gram negative bacteria, the present method also provides a persistent viral control.

The method further exhibits a log reduction against acid-labile viruses, including rhinovirus serotypes of about 4 after 30 seconds contact, and a log reduction against these acid-labile viruses of at least 3 about five hours after contact, and at least about 2 about six to about eight hours after skin contact with a suitable compound or composition. The method also is mild, and it is not necessary to rinse or wipe the compound or composition from the skin.

The following compounds are capable of sufficiently lowering skin pH in accordance with the method of the present invention.

### A. Organic Acid

A present method can utilize an organic acid in a sufficient amount to reduce a surface pH to less than about 4, and thereby control and inactivate bacteria and viruses on a surface contacted by the organic acid. The organic acid helps provide a rapid control of acid-labile viruses, and provides a persistent viral control.

Upon application to a surface, such as human skin, the pH of the surface is sufficiently lowered such that a persistent viral control is achieved. In preferred embodiments, a residual amount of the organic acid remains on the skin, even after a rinsing step, in order to impart a persistent viral control. However, even if the organic acid is essentially completely rinsed from the surface, the surface pH has been sufficiently lowered to impart a viral control for at least 0.5 hours.

In particular, an organic acid is applied to a surface in a sufficient amount such that the pH of the animate or inanimate surface contacted by the organic acid is lowered to degree wherein a persistent viral control is achieved, i.e., to less than about 4. This persistent viral control is achieved regardless of whether the organic acid is rinsed from, or allowed to remain on, the contacted surface. The organic acid remains at least partially undissociated after application, and remains so when diluted, or during application and rinsing.

The organic acid has a pKa of about 1 to about 6, and preferably about 2 to about 5.5. To achieve the full advantage of the present invention, the organic acid has a pKa of about 2.5 to about 5. Such organic acids have a sufficient acid strength to reduce a surface pH to less than about 4. Preferably, the organic acid is substantive to the treated surface to enhance the persistent antimicrobial properties.

Typically, an organic acid is included in a composition in an amount of about 0.05% to about 6%, and preferably about 0.1% to about 5%, by weight of the composition. To achieve the full advantage, the organic acid is present in a composition in an amount of about 0.15% to about 4%, by weight of the composition. The amount of organic acid is related to the class of organic acid used, and to the identity of the specific acid or acids used.

An organic acid useful in a present method comprises a monocarboxylic acid, a polycarboxylic acid, a polymeric acid having a plurality of carboxylic, phosphate, sulfonate, and/or sulfate moieties, or mixtures thereof. In addition to acid moieties, the organic acid also can contain other moieties, for example, hydroxy groups and/or amino groups. In addition, an organic acid anhydride can be used in the present method as the organic acid.

In one embodiment, the organic acid comprises a monocarboxylic acid having a structure RCO₂H, wherein R is C₁₋₃alkyl, hydroxyC₁₋₃alkyl, haloC₁₋₃alkyl, phenyl, or substituted phenyl. The monocarboxylic acid preferably has a water solubility of at least about 0.05%, by weight, at 25° C. The alkyl groups can be substituted with phenyl groups and/or phenoxy groups, and these phenyl and phenoxy groups can be substituted or unsubstituted.

Nonlimiting examples of monocarboxylic acids useful in the present invention are acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof. Additional substituted benzoic acids are disclosed in U.S. Pat. No. 6,294,186, incorporated herein by reference. Examples of substituted benzoic acids include, but are not limited to, salicyclic acid, 2-nitrobenzoic acid, thiosalicylic acid, 2,6-dihydroxybenzoic acid, 5-nitrosalicyclic acid, 5-bromosalicyclic acid, 5-iodosalicyclic acid, 5-fluorosalicylic acid, 3-chlorosalicylic acid, 4-chlorosalicyclic acid, and 5-chlorosalicyclic acid.

In another embodiment, the organic acid comprises a polycarboxylic acid. The polycarboxylic acid contains at least two, and up to four, carboxylic acid groups. The polycarboxylic acid also can contain hydroxy or amino groups, in addition to substituted and unsubstituted phenyl groups. Preferably, the polycarboxylic acid has a water solubility of at least about 0.05%, by weight, at 25° C.

Nonlimiting examples of polycarboxylic acids useful in the present invention include malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

Anhydrides of polycarboxylic and monocarboxylic acids also are organic acids useful in the present compositions. Preferred anhydrides are anhydrides of polycarboxylic acids. At least a portion of the anhydride is hydrolyzed to a carboxylic acid because of the pH of the composition. It is envisioned that an anhydride can be slowly hydrolyzed on a surface contacted by the composition, and thereby assist in providing a persistent antiviral activity.

In a third embodiment, the organic acid comprises a polymeric carboxylic acid, a polymeric sulfonic acid, a sulfated polymer, a polymeric phosphoric acid, or mixtures thereof. The polymeric acid has a molecular weight of about 500 g/mol to 10,000,000 g/mol, and includes homopolymers, copolymers, and mixtures thereof. The polymeric acid preferably is capable of forming a substantive film on a skin surface and has a pKa less than about 6, preferably less than about 5.5, and a glass transition temperature, T_{g}, of less than about 25° C., preferably less than about 20° C., and more preferably less than about 15° C. The glass transition temperature is the temperature at which an amorphous material, such as a polymer, changes from a brittle vitreous state to a plastic state. The T_{g} of a polymer is readily determined by persons skilled in the art using standard techniques.

The polymeric acids are uncrosslinked or only very minimally crosslinked. The polymeric acids therefore are water soluble or at least water dispersible. The polymeric acids typically are prepared from ethylenically unsaturated monomers having at least one hydrophilic moiety, such as carboxyl, carboxylic acid anhydride, sulfonic acid, and sulfate.

Examples of monomers used to prepare the polymeric organic acid include, but are not limited to:

(a) Carboxyl group-containing monomers, e.g., monoethylenically unsaturated mono- or polycarboxylic acids, such as acrylic acid, methacrylic acid, maleic acid, fumaric acid, crotonic acid, sorbic acid, itaconic acid, ethacrylic acid, o-chloroacrylic acid, α-cyanoacrylic acid, β-methlacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, citraconic acid, mesaconic acid, glutaconic acid, aconitic acid, tricarboxyethylene, and cinnamic acid;

(b) Carboxylic acid anhydride group-containing monomers, e.g., monoethylenically unsaturated polycarboxylic acid anhydrides, such as maleic anhydride; and

(c) Sulfonic acid group-containing monomers, e.g., aliphatic or aromatic vinyl sulfonic acids, such as vinylsulfonic acid, allylsulfonic acid, vinyltoluenesulfonic acid, styrenesulfonic acid, sulfoethyl(meth)acrylate, 2-acrylamido-2-methylpropane sulfonic acid, sulfopropyl (meth)acrylate, and 2-hydroxy-3-(meth)acryloxy propyl sulfonic acid.

The polymeric acid can contain other copolymerizable units, i.e., other monoethylenically unsaturated comonomers, well known in the art, as long as the polymer is substantially, i.e., at least 10%, and preferably at least 25%, acid group containing monomer units. To achieve the full advantage of the present invention, the polymeric acid contains at least 50%, and more preferably, at least 75%, and up to 100%, acid group containing monomer units. The other copolymerizable units, for example, can be styrene, an alkyl acrylate, or an alkyl methacrylate. The polymeric acid also can be partially neutralized, which assists dispersion of the polymeric acid into a composition. However, a sufficient number of the acid groups remain unneutralized to reduce skin pH and impart a persistent antiviral activity.

One preferred polymeric acid is a polyacrylic acid, either a homopolymer or a copolymer, for example, a copolymer of acrylic acid and an alkyl acrylate and/or alkyl methacrylate. Another preferred polymeric acid is a homopolymer or a copolymer of methacrylic acid.

Exemplary polymeric acids useful in the present invention include, but are not limited to:

| | |
|---|---|
| Carbomers | (CARBOPOL 910, |
| | 934, 934P, 940, |
| | 941, ETD 2050; |
| | ULTREZ 10, 21) |
| Acrylates/C20-30 Alkyl Acrylate Crosspolymer | (ULTREZ 20) |
| Acrylates/Beheneth 25 Methacrylate Copolymer | (ACULYN 28) |
| Acrylates/Steareth 20 Methacrylate Copolymer | (ACULYN 22) |
| Acrylates/Steareth 20 Methacrylate | (ACULYN 88) |
| Crosspolymer | |
| Acrylates Copolymer | (CAPIGEL 98) |
| Acrylates Copolymer | (AVALURE AC) |
| Acrylates/Palmeth 25 Acrylate Copolymer | (SYNTHALEN 2000) |
| Ammonium Acrylate Copolymers | |
| Sodium Acrylate/Vinyl Alcohol Copolymer | |
| Sodium polymethacrylate | |
| Acrylamidopropyltrimonium Chloride/Acrylates | |
| Copolymer | |
| Acrylates/Acrylamide Copolymer | |
| Acrylates/Ammonium Methacrylate Copolymer | |
| Acrylates/C10-30 Alkyl Acrylate Crosspolymer | |
| Acrylates/Diacetoneacrylamide Copolymer | |
| Acrylates/Octylacrylamide Copolymer | |
| Acrylates/VA Copolymer | |
| Acrylic Acid/Acrylonitrogens Copolymer | |

In a preferred embodiment of the present invention, the organic acid comprises one or more polycarboxylic acid, e.g., citric acid, malic acid, tartaric acid, or a mixture of any two or all three of these acids, and a polymeric acid containing a plurality of carboxyl groups, for example, homopolymers and copolymers of acrylic acid or methacrylic acid.

### B. Inorganic Acid

The present method also can utilize an inorganic acid that is noncorrosive to the skin, in lieu of or together with an organic acid. Preferably, the inorganic acid is substantive to the surface to which it is applied. Like the organic acid, an inorganic acid typically is present in a composition for application to the skin in an amount of about 0.05% to about 6%, and preferably about 0.1% to about 5%, by weight of the composition. To achieve the full advantage of the present invention, the inorganic acid is present in an amount of about 0.15% to about 4%, by weight of the composition.

The inorganic acid has a pKa at 25° C. of less than 6, and preferably less than 5.5. To achieve the full advantage of the present invention, the inorganic acid has a pKa of 25° C. of less than 5. The identity of the inorganic acid is not limited, but the inorganic acid must possess sufficient acidity to lower a surface pH to less than about 4 without adversely effecting the surface, e.g., corrosion of an inanimate surface or irritation of an animate surface. Examples of inorganic acids include, but are not limited to, phosphoric acid, pyrophosphoric acid, polyphosphoric acid, phosphorous acid, and mixtures thereof, and similar noncorrosive inorganic acids.

### C. Inorganic Salt

An inorganic salt comprising a cation having a valence of 2, 3, or 4 and a counterion capable of lowering a surface pH, such as a skin pH, to less than about 4 can be used in lieu of, or together with, an organic acid and/or an inorganic acid. The inorganic salt, alone or in combination with the organic acid and/or inorganic acid, is present in a sufficient amount to control and inactivate viruses on a surface contacted in accordance with the present invention. Like the organic acid and inorganic acid, the inorganic salt provides a rapid control of acid-labile viruses, and provides a persistent viral control, by reducing the skin pH to less than about 4.

A cation of the inorganic salt has a valence of 2, 3, or 4, and can be, for example, magnesium, calcium, barium, aluminum, iron, cobalt, nickel, copper, zinc, zirconium, and tin. Preferred cations include, for example, zinc, aluminum, and copper.

Anions of the inorganic salt include, but are not limited to, bisulfate, sulfate, dihydrogen phosphate, monohydrogen phosphate, halides, such as chloride, iodide, and bromide, and nitrate. Preferred inorganic salts include chlorides and dihydrogen phosphates.

An inorganic salt is used in accordance with the present method in an amount of about 0.1% to about 5%, and preferably about 0.2% to about 2%, by weight of a composition. To achieve the full advantage of the present invention, the inorganic salt is applied to a surface as an aqueous solution containing about 0.3% to about 1% of an inorganic salt, by weight of the composition.

In one nonlimiting embodiment, the inorganic salt comprises a divalent zinc salt. A divalent zinc salt is described in detail herein, but it should be understood that similar polyvalent metal salts similarly can be used in accordance with the present method. In particular, divalent zinc salts useful in the present invention can have an organic or an inorganic counterion. The counterion reduces the skin pH to less than about 4. In preferred embodiments, the divalent zinc ion, or any other useful cation, is applied in an unchelated or uncomplexed form, which allows the cation to more effectively contact, and potentially deposit, on the skin to assist in an effective and persistent control of microbes.

In some embodiments, however, an organic counterion complexes with the divalent zinc ion, i.e., Zn⁺². Such embodiments are useful as long as the counterion lowers skin pH to less than about 4, and preferably the complexed Zn⁺² has a sufficient equilibrium amount of uncomplexed Zn⁺² help effectively control microbes on the skin.

A preferred divalent zinc salt, or other useful inorganic salt, has a water solubility of at least about 0.1 g (grams) per 100 ml (milliliters) of water at 25° C., and preferably about 0.25 g/100 ml of water at 25° C. Water-insoluble forms of zinc, e.g., zinc oxide, are not useful because the counterion is incapable of lowering skin pH and the zinc ion is essentially unavailable to assist in controlling microbes on the skin.

In most preferred embodiments, the divalent zinc salt, or other useful inorganic salt, is water soluble, but resists rinsing from the skin to provide a persistent virucidal efficacy. Therefore, in most preferred embodiments, the counterion effectively lowers skin pH for about four hours or more and the divalent zinc or other cation is substantive to the skin, regardless of whether the aqueous solution containing the inorganic salt is rinsed from the skin after application, or is allowed to remain on the skin after application.

Although prior compositions including zinc salts addressed the ability of zinc ions to disrupt viral replication when the virus enters the epithelial cells of the nasal, oral, and pharyngeal mucosa, thus shortening the duration of the common cold, the present invention is directed to the surprising discovery that suitable inorganic salts, including zinc salts, provide unexpected benefits in protecting individuals from rhinoviral infection when applied to the skin, especially the hands. The benefit of preventing a viral infection therefore provides a level of protection greater than simply shortening the duration of infection.

Zinc salts useful in a present antimicrobial composition include, but are not limited to, divalent zinc salts having a counterion selected from the group consisting of gluconate, acetate, chloride, bromide, citrate, formate, glycerophosphate, iodide, lactate, salicylate, tartrate, and mixtures thereof.

### D. Aluminum, Zirconium, and Aluminum-Zirconium Complexes

An aluminum, zirconium, or aluminum-zirconium complex can be used in lieu of, or together with, an organic acid, an inorganic acid, and/or an inorganic salt. Such a complex, alone or in combination with an organic acid, an inorganic acid, and/or an inorganic salt, is applied to a surface in a sufficient amount to reduce skin pH to less than about 4, and thereby control and inactivate viruses on the surface. Like the organic acid, the inorganic acid, and the inorganic salt, these complexes provide a rapid control of acid-labile viruses, and can provide a persistent virus control for about four hours or more after application to the skin.

The aluminum, zirconium, and aluminum-zirconium complexes typically are polymeric in nature, contain hydroxyl moieties, and have an anion such as, but not limited to sulfate, chloride, chlorohydroxide, alumformate, lactate, benzyl sulfonate, or phenyl sulfonate. Exemplary classes of useful complexes include, but are not limited to, aluminum hydroxyhalides, zirconyl oxyhalides, zirconyl hydroxyhalides, and mixtures thereof. These complexes typically are acidic in nature, thereby providing a composition having a pH less than about 5 and typically having a pH of about 2 to about 4.5, and preferably about 3 to about 4.5. Accordingly, the complexes are capable of lowering skin pH to less than about 4.

Exemplary aluminum compounds include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts, also referred to as zirconyl salts and zirconyl hydroxy salts, and represented by the general empirical formula ZrO(OH)_{2-nz}-L_{z}, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof.

Exemplary complexes, therefore, include, but are not limited to, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, and mixtures thereof. Numerous other useful compounds are listed in WO 91/19222 and in the CTFA Cosmetic Ingredient Handbook, The Cosmetic, Toiletry and Fragrance Association, Inc., Washington, D.C., p. 56, 1988, hereinafter the CTFA Handbook, incorporated herein by reference.

Preferred compounds are the aluminum-zirconium chlorides complexed with an amino acid, like glycine, and the aluminum chlorohydrates. Preferred aluminum-zirconium chloride glycine complexes have an aluminum (Al) to zirconium (Zr) ratio of about 1.67 to about 12.5, and a total metal (Al+Zr) to chlorine ratio (metal to chlorine) of about 0.73 to about 1.93.

Typically, the present method is performed by incorporating an organic acid, inorganic acid, inorganic salt, zinc and/or aluminum complex, or mixtures thereof into a composition, then applying the composition to a surface. The carrier for the organic acid, inorganic acid, inorganic salt, and zinc and/or aluminum complex in such a composition comprises water. The composition can be a rinse-off or leave-on composition, as long as the surface contacted has a pH of less than about 4.

In accordance with the invention, a composition useful in the present method for lowering skin pH can contain various optional ingredients described hereafter, such as antimicrobial agents, disinfecting alcohols, hydrotropes, polyhydric solvents, gelling agents, pH adjusters, vitamins, dyes, skin conditioners, and perfumes.

The pH of a composition for lowering skin pH preferably is less than about 5, and preferably less than about 4.5. To achieve the full advantage of the present invention, the pH is less than about 4. Typically, the pH of a composition for lowering skin pH is about 2 to less than about 5, and preferably about 2.5 to about 4.5.

### Optional Ingredients

### Antimicrobial Agent

An antimicrobial agent can be present, if at all, in a composition for lowering skin pH in an amount of 0.1% to about 5%, and preferably about 0.1% to about 2%, and more preferably, about 0.3% to about 1%, by weight of the composition.

Optional antimicrobial agents useful in the present invention are exemplified by the following classes of compounds used alone or in combination:

(1) Phenolic Antimicrobial Agents

(a) 2-Hydroxydiphenyl Compounds

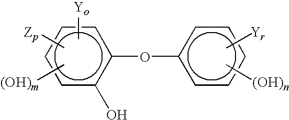

wherein Y is chlorine or bromine, Z is SO₃H, NO₂, or C₁-C₄ alkyl, r is 0 to 3, o is 0 to 3, p is 0 or l, m is 0 or 1, and n is 0 or 1.

In preferred embodiments, Y is chlorine or bromine, m is 0, n is 0 or 1, o is 1 or 2, r is 1 or 2, and p is 0.

In especially preferred embodiments, Y is chlorine, m is 0, n is 0, o is 1, r is 2, and p is 0.

A particularly useful 2-hydroxydiphenyl compound has a structure:

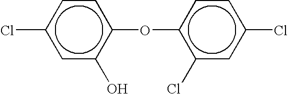

having the adopted name, triclosan, and available commercially under the tradename IRGASAN DP300, from Ciba Specialty Chemicals Corp., Greensboro, N.C. Another useful 2-hydroxydiphenyl compound is 2,2′-dihydroxy-5,5′-dibromo-diphenyl ether.

(b) Phenol Derivatives

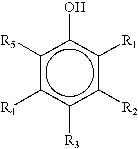

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl; and R₅ is hydro or nitro. Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsulfonic acid. Other phenol derivatives are listed in U.S. Pat. No. 6,436,885, incorporated herein by reference.

(c) Diphenyl Compounds

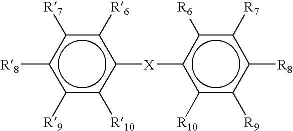

wherein X is sulfur or a methylene group, R₆, and R′₆ are hydroxy, and R₇, R′₇, R₈, R′₈, R₉, R′₉, R₁₀, and R′₁₀, independent of one another, are hydro or halo. Specific, nonlimiting examples of diphenyl compounds are hexachlorophene, tetrachlorophene, dichlorophene, 2,3-dihydroxy-5,5′-dichlorodiphenyl sulfide, 2,2′-dihydroxy-3,3′,5,5′-tetrachlorodiphenyl sulfide, 2,2′-dihydroxy-3,5′,5,5′,6,6′-hexachlorodiphenyl sulfide, and 3,3′-dibromo-5,5′-dichloro-2,2′-dihydroxydiphenylamine. Other diphenyl compounds are listed in U.S. Pat. No. 6,936,885, incorporated herein by reference.

(2) Quaternary Ammonium Antimicrobial agents

Useful quaternary ammonium antibacterial agents have a general structural formula:

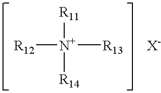

wherein at least one of R₁₁, R₁₂, R₁₃, and R₁₄, is an alkyl, aryl, or alkaryl substituent containing 6 to 26 carbon atoms. Alternatively, any two of the R substituents can be taken together, with the nitrogen atom, to form a five- or six-membered aliphatic or aromatic ring. Preferably, the entire ammonium cation portion of the antibacterial agent has a molecular weight of at least 165.

The substituents R₁₁, R₁₂, R₁₃, and R₁₄ can be straight chained or can be branched, but preferably are straight chained, and can include one or more amide, ether, or ester linkage. In particular, at least one substituent is C₆-C₂₆alkyl, C₆-C₂₆alkoxyaryl, C₆-C₂₆alkaryl, halogen-substituted C₆-C₂₆alkaryl, C₆-C₂₆alkylphenoxyalkyl, and the like. The remaining substituents on the quaternary nitrogen atom other than the above-mentioned substituent typically contain no more than 12 carbon atoms. In addition, the nitrogen atom of the quaternary ammonium antibacterial agent can be present in a ring system, either aliphatic, e.g., piperidinyl, or aromatic, e.g., pyridinyl. The anion X can be any salt-forming anion which renders the quaternary ammonium compound water soluble. Anions include, but are not limited to, a halide, for example, chloride, bromide, or iodide, methosulfate, and ethosulfate.

Preferred quaternary ammonium antimicrobial agents have a structural formula:

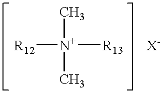

wherein R₁₂ and R₁₃, independently, are C₈-C₁₂alkyl, or R₁₂ is C₁₂-C₁₆alkyl, C₈-C₁₈alkylethoxy, or C₈-C₁₈alkylphenylethoxy, and R₁₃ is benzyl, and X is halo, methosulfate, ethosulfate, or p-toluenesulfonate. The alkyl groups R₁₂ and R₁₃ can be straight chained or branched, and preferably are linear.

The quaternary ammonium antimicrobial agent in a present composition can be a single quaternary ammonium compound, or a mixture of two or more quaternary ammonium compounds. Particularly useful quaternary ammonium antimicrobial agents include dialkyl(C₈-C₁₀) dimethyl ammonium chlorides (e.g., dioctyl dimethyl ammonium chloride), alkyl dimethyl benzyl ammonium chlorides (e.g., benzalkonium chloride and myristyl dimethylbenzyl ammonium chloride), alkyl methyl dodecyl benzyl ammonium chloride, methyl dodecyl xylene-bis-trimethyl ammonium chloride, benzethonium chloride, dialkyl methyl benzyl ammonium chloride, alkyl dimethyl ethyl ammonium bromide, and an alkyl tertiary amine. Polymeric quaternary-ammonium compounds based on these monomeric structures also can be used in the present invention. One example of a polymeric quaternary ammonium compound is POLYQUAT®, e.g., a 2-butenyl dimethyl ammonium chloride polymer. The above quaternary ammonium compounds are available commercially under the tradenames BARDAC®, BTC®, HYAMINE®, BARQUAT®, and LONZABAC®, from suppliers such as Lonza, Inc., Fairlawn, N.J. and Stepan Co., Northfield, Ill.

Additional examples of quaternary ammonium antimicrobial agents include, but are not limited to, alkyl ammonium halides, such as cetyl trimethyl ammonium bromide; alkyl aryl ammonium halides, such as octadecyl dimethyl benzyl ammonium bromide; N-alkyl pyridinium halides, such as N-cetyl pyridinium bromide; and the like. Other suitable quaternary ammonium antimicrobial agents have amide, ether, or ester moieties, such as octylphenoxyethoxy ethyl dimethyl benzyl ammonium chloride, N-(laurylcocoaminoformylmethyl)pyridinium chloride, and the like. Other classes of quaternary ammonium antimicrobial agents include those containing a substituted aromatic nucleus, for example, lauryloxyphenyl trimethyl ammonium chloride, cetylaminophenyl trimethyl ammonium methosulfate, dodecylphenyl trimethyl ammonium methosulfate, dodecylbenzyl trimethyl ammonium chloride, chlorinated dodecylbenzyl trimethyl ammonium chloride, and the like.

Specific quaternary ammonium antimicrobial agents include, but are not limited to, behenalkonium chloride, cetalkonium chloride, cetarylalkonium bromide, cetrimonium tosylate, cetyl pyridinium chloride, lauralkonium bromide, lauralkonium chloride, lapyrium chloride, lauryl pyridinium chloride, myristalkonium chloride, olealkonium chloride, and isostearyl ethyldimonium chloride. Preferred quaternary ammonium antimicrobial agents include benzalkonium chloride, benzethonium chloride, cetyl pyridinium bromide, and methylbenzethonium chloride.

(3) Anilide and Bisguanidine Antimicrobial Agents

Useful anilide and bisguanadine antimicrobial agents include, but are not limited to, triclocarban, carbanilide, salicylanilide, tribromosalan, tetrachlorosalicylanilide, fluorosalan, chlorhexidine gluconate, chlorhexidine hydrochloride, and mixtures thereof.

### Disinfecting Alcohol

Compositions useful in the present method for lowering skin pH to produce a persistent control of bacteria and viruses also can contain, if at all, 10% to about 90%, by weight of an optional disinfecting alcohol. Preferred compositions contain an optional disinfecting alcohol in an amount of about 10% to about 70%, and more preferably about 20% to about 65%, by weight.

As used herein, the term “disinfecting alcohol” is a water-soluble alcohol containing one to six carbon atoms. Disinfecting alcohols include, but are not limited to, methanol, ethanol, propanol, and isopropyl alcohol.

### Other Optional Ingredients

A composition for lowering skin pH useful in the present method also can contain other optional ingredients well known to persons skilled in the art. Such optional ingredients are present in a sufficient amount to perform their intended function and not adversely affect the efficacy of the composition. Optional ingredients typically are present, collectively, from 0% to about 50%, by weight of the composition.

Classes of optional ingredients include, but are not limited to, surfactants, hydrotropes, polyhydric solvents, gelling agents, dyes, fragrances, pH adjusters, thickeners, viscosity modifiers, chelating agents, skin conditioners, emollients, preservatives, buffering agents, foam stabilizers, antioxidants, foam enhancers, chelating agents, opacifiers, and similar classes of optional ingredients known to persons skilled in the art.

A surfactant can be included in a composition for lowering skin pH, if at all, in an amount of 0.1% to about 15%, and typically 0.1% to about 10%, by weight, of the composition. More typically, if present at all, the composition contains 0% to about 7%, by weight of the surfactant. The optional surfactant is stable at the pH of the composition and is compatible with the other ingredients present in the composition.

The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

The compositions, therefore, can contain an anionic surfactant having a hydrophobic moiety, such as a carbon chain including about 8 to about 30 carbon atoms, and particularly about 12 to about 20 carbon atoms, and further has a hydrophilic moiety, such as sulfate, sulfonate, carbonate, phosphate, or carboxylate. Often, the hydrophobic carbon chain is etherified, such as with ethylene oxide or propylene oxide, to impart a particular physical property, such as increased water solubility or reduced surface tension to the anionic surfactant.

Suitable anionic surfactants include, but are not limited to, compounds in the classes known as alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylaryl sulfonates, alkyl monoglyceride sulfates, alkyl monoglyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates, acyl glutamates, alkyl sulfoacetates, acylated peptides, acyl lactylates, anionic fluoro surfactants, and mixtures thereof. Additional anionic surfactants are listed in McCutcheon's Emulsifiers and Detergents, 1993 Annuals, (hereafter McCutcheon's), Mccutcheon Division, MC Publishing Co., Glen Rock, N.J., pp. 263-266, incorporated herein by reference. Numerous other anionic surfactants, and classes of anionic surfactants, are disclosed in U.S. Pat. No. 3,929,678 and U.S. Patent Publication No. 2002/0098159, each incorporated herein by reference.

Specific, nonlimiting classes of anionic surfactants useful in the present invention include, but are not limited to, a C₁-C₁₈ alkyl sulfonate, a C₈-C₁₈ alkyl sulfate, a C₈-C₁₈ fatty acid salt, a C₈-C₁₈ alkyl ether sulfate having one or two moles of ethoxylation, a C₁-C₁₈ alkamine oxide, a C₈-C₁₈ alkoyl sarcosinate, a C₈-C₁₈ sulfoacetate, a C₈-C₁₈ sulfosuccinate, a C₈-C₁₈ alkyl diphenyl oxide disulfonate, a C₈-C₁₈ alkyl carbonate, a C₈-C₁₈ alpha-olefin sulfonate, a methyl ester sulfonate, and mixtures thereof. The C₈-C₁₈ alkyl group contains eight to eighteen carbon atoms, and can be straight chain (e.g., lauryl) or branched (e.g., 2-ethylhexyl). The cation of the anionic surfactant can be an alkali metal (preferably sodium or potassium), ammonium, —C₁-C₄ alkylammonium (mono-, di-, tri-), or C₁-C₃ alkanolammonium (mono-, di-, tri-). Lithium and alkaline earth cations (e.g., magnesium) can be used, but are not preferred.

Specific surfactants include, but are not limited to, lauryl sulfates, octyl sulfates, 2-ethylhexyl sulfates, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, ricinoleates, cetyl sulfates, and similar surfactants. Additional examples of surfactants can be found in “CTFA Cosmetic Ingredient Handbook,” J. M. Nikitakis, ed., The Cosmetic, Toiletry and Fragrance Association, Inc., Washington, D.C. (1988) (hereafter CTFA Handbook), pages 10-13, 42-46, and 87-94, incorporated herein by reference.

The compositions also can contain nonionic surfactants. Typically, a nonionic surfactant has a hydrophobic base, such as a long chain alkyl group or an alkylated aryl group, and a hydrophilic chain comprising a sufficient number (i.e., 1 to about 30) of ethoxy and/or propoxy moieties. Examples of classes of nonionic surfactants include ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, and mixtures thereof.

Exemplary nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, and mixtures thereof.

Numerous other nonionic surfactant's are disclosed in McCutcheon's, at pages 1-246 and 266-272; in the CTFA International Cosmetic Ingredient Dictionary, Fourth Ed., Cosmetic, Toiletry and Fragrance Association, Washington, D.C. (1991) (hereinafter tire CTFA Dictionary) at pages 1-651; and in the CTFA Handbook, at pages 16-94, each incorporated herein by reference.

In addition to anionic and nonionic surfactants, cationic, ampholytic, and amphoteric surfactants can be used in the compositions. Useful cationic surfactants include those having a structural formula

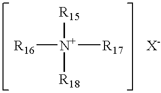

wherein R₁₅ is an alkyl group having about 12 to about 30 carbon atoms, or an aromatic, aryl, or alkaryl group having about 12 to about 30 carbon atoms; R₁₆, R₁₇, and R₁₈, independently, are selected from the group consisting of hydrogen, an alkyl group having 1 to about 22 carbon atoms, or aromatic, aryl, or alkaryl groups having from about 12 to about 22 carbon atoms; and X is a compatible anion, preferably selected from the group consisting of chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁₅, R₁₆, R₁₇, and R₁₈ also can contain ester and/or ether linkages, or hydroxy or amino group substituents (e.g., the alkyl groups can contain polyethylene glycol and polypropylene glycol moieties).

Preferably, R₁₅ is an alkyl group having about 12 to about 22 carbon atoms; R₁₆ is H or an alkyl group having 1 to about 22 carbon atoms; and R₁₇ and R₁₈, independently are H or an alkyl group having 1 to about 3 carbon atoms. More preferably, R₃₅ is an alkyl group having about 12 to about 22-carbon atoms, and R₁₆, R₁₇, and R₁₈ are H or an alkyl group having 1 to about 3 carbon atoms.

Other useful cationic surfactants include amino-amides, wherein in the above structure R₁₀ alternatively is R₁₉CONH—(CH₂)ₙ, wherein R₁₉, is an alkyl group having about 12 to about 22 carbon atoms, and n is an integer of 2 to 6, more preferably 2 to 4, and most preferably 2 to 3. Nonlimiting examples of these cationic surfactants include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof.

Additional quaternary ammonium salts include those wherein the C₁₂-C₃₀ alkyl carbon chain is derived from a tallow fatty acid or from a coconut fatty acid. The term “tallow” refers to an alkyl group derived from tallow fatty acids (usually hydrogenated tallow fatty acids), which generally has mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, and mixtures thereof. An example of a quaternary ammonium compound having an alkyl group with an ester linkage is ditallowyl oxyethyl dimethyl ammonium chloride.

Ampholytic surfactants, i.e., amphoteric and zwitterionic surfactants, can be broadly described as derivatives of secondary and tertiary amines having straight chain or branched aliphatic radicals, and wherein one of the aliphatic substituents contains from about 8 to about 18 carbon atoms and at least one of the aliphatic substituents contains an anionic water-solubilizing group, e.g., carboxy, sulfonate, or sulfate.

More particularly, one class of ampholytic surfactants include sarcosinates and taurates having the general structural formula

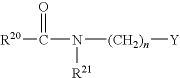

wherein R²⁰ is C₁₁-C₂₁ alkyl, R²¹ is hydrogen or C₁-C₂ alkyl, Y is CO₂M or SO₃M, M is an alkali metal, and n is a number 1 through 3.

Another class of ampholytic surfactants is the amide sulfosuccinates having the structural formula

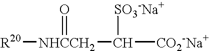

The following classes of ampholytic surfactants also can be used:

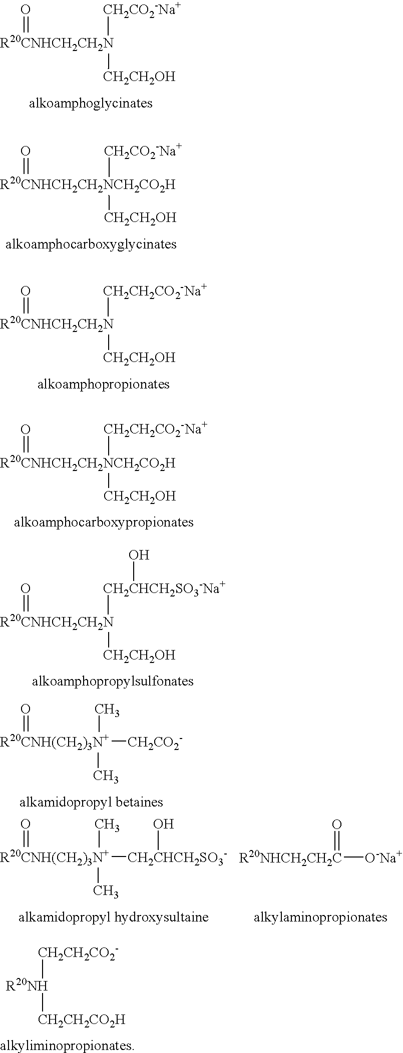

Additional classes of ampholytic surfactants include the phosphobetaines and the phosphitaines.

Specific, nonlimiting examples of ampholytic surfactants useful in the present invention are sodium coconut N-methyl taurate, sodium oleyl N-methyl taurate, sodium tall oil acid N-methyl taurate, sodium palmitoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamidobis-(2-hydroxyethyl)propylsultaine, disodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, caprvioamphocarboxypropionate, dihydroxyethyl tallow glycinate, cocamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, lauric myristic amido propyl monosodium phosphitaine, and mixtures thereof.

Useful amphoteric surfactants also include the amine oxides. Amine oxides have a general structural formula wherein the hydrophilic portion contains a nitrogen atom that is bound to an oxygen atom with a semipolar bond.

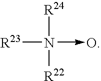

R²², R²³, and R²⁴ can be a saturated or unsaturated, branched, or unbranched alkyl or alkenyl group having 1 to about 24 carbon atoms. Preferred amine oxides contain at least one R group that is an alkyl chain of 8 to 22 carbon atoms. Nonlimiting examples of amine oxides include alkyl dimethyl amine oxides, such as decylamine oxide, cocamine oxide, myristamine oxide, and palmitamine oxide. Also useful are the alkylaminopropylamine oxides, for example, coamidopropylamine oxide and stearamidopropylamine oxide.

Nonlimiting examples of preferred surfactants utilized in a composition include those selected from the group consisting of alkyl sulfates; alkyl ether sulfates; alkyl benzene sulfonates; alpha olefin sulfonates; primary or secondary alkyl sulfonates; alkyl phosphates; acyl taurates; alkyl sulfosuccinates; alkyl sulfoacetates; sulfonated fatty acids; alkyl trimethyl ammonium chlorides and bromides; dialkyl dimethyl ammonium chlorides and bromides; alkyl dimethyl amine oxides; alkylamidopropyl amine oxides; alkyl betaines; alkyl amidopropyl betaines; and mixtures thereof. More preferred surfactants include those; selected from the group consisting of alkyl sulfates; alkyl ether sulfates; alkyl benzene sulfonates; alpha olefin sulfonates; primary or secondary alkyl sulfonates; alkyl dimethyl amine oxides; alkyl betaines; and mixtures thereof.

A hydrotrope, if present at all, is present in an amount of 0% to about 30%, and preferably 0% to about 20%, by weight of the composition. More preferably, a composition contains 0% to about 15%, by weight of a hydrotrope.

A hydrotrope is a compound that has an ability to enhance the water solubility of other compounds. A hydrotrope utilized in the present invention lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A polyhydric solvent, if present at all, is present in an amount of 0% to about 30%, and preferably 0% to about 20%, by weight of the composition. In contrast to a disinfecting alcohol, a polyhydric solvent contributes minimally, if at all, to the efficacy of the composition.

The term “polyhydric solvent” as used herein is a water-soluble organic compound containing two to six, and typically two or three, hydroxyl groups. The term “water-soluble” means that the polyhydric solvent has a water solubility of at least 0.1 g of polyhydric solvent per 100 g of water at 25° C. There is no upper limit to the water solubility of the polyhydric solvent, e.g., the polyhydric solvent and water can be soluble in all proportions.

The term polyhydric solvent, therefore, encompasses water-soluble diols, triols, and polyols. Specific examples of hydric solvents include, but are not limited to, ethylene glycol, propylene glycol, glycerol, diethylene glycol, dipropylene glycol, tripropylene glycol, hexylene glycol, butylene glycol, 1,2,6-hexanetriol, sorbitol, PEG-4, and similar polyhydroxy compounds.

Other specific classes of optional ingredients include alkanolamides as foam boosters and stabilizers; inorganic phosphates, sulfates, and carbonates as buffering agents; EDTA and phosphates as chelating agents; and acids and bases as pH adjusters.

Examples of preferred classes of optional basic pH adjusters are ammonia; mono-, di-, and tri-alkyl amines; mono-, di-, and tri-alkanolamines; alkali metal and alkaline earth metal hydroxides; and mixtures thereof. However, the identity of the basic pH adjuster is not limited, and any basic pH adjuster known in the art can be used. Specific, nonlimiting examples of basic pH adjusters are ammonia; sodium, potassium, and lithium hydroxide; monoethanolamine; triethylamine; isopropanolamine; diethanolamine; and triethanolamine.

Examples of preferred classes of optional acidic pH adjusters are the mineral acids. Nonlimiting examples of mineral acids are hydrochloric acid, nitric acid, phosphoric acid, and sulfuric acid. The identity of the acidic pH adjuster is not limited and any acidic pH adjuster known in the art, alone or in combination, can be used.

An optional alkanolamide to provide composition thickening can be, but is not limited to, cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. Alkanolamides are noncleansing surfactants and are added, if at all, in small amounts to thicken the composition.

The compositions also can contain, if at all, about 0.1% to about 5%, by weight, and preferably 0.1% to about 3%, by weight of an optional gelling agent. More preferably, the compositions contain about 0.1% to about 2.5%, by weight of a gelling agent. The compositions contain a sufficient amount of gelling agent such that the composition is a viscous liquid, gel, or semisolid that can be easily applied to, and rubbed on, the skin or other surface. Persons skilled in the art are aware of the type and amount of gelling agent to include in the composition to provide the desired composition viscosity or consistency.

The term “gelling agent” as used here and hereafter refers to a compound capable of increasing the viscosity of a water-based composition, or capable of converting a water-based composition to a gel or semisolid. The gelling agent, therefore, can be organic in nature, for example, a natural gum or a synthetic polymer, or can be inorganic in nature.

The following are nonlimiting examples of gelling agents that can be used in the present intention. In particular, the following compounds, both organic and inorganic, act primarily by thickening or gelling the aqueous portion of the composition:

acacia, agar, algin, alginic acid, ammonium alginate, ammonium chloride, ammonium sulfate, amylopectin, attapulgite, bentonite, C₉₋₅ alcohols, calcium acetate, calcium alginate, calcium carrageenan, calcium chloride, caprylic alcohol, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium-trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyvinyl alcohol, potassium alginate, potassium carrageenan, potassium chloride, potassium sulfate, potato starch, propylene glycol alginate, sodium carboxymethyl dextran, sodium carrageenan, sodium cellulose sulfate, sodium chloride, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof.

The following additional nonlimiting examples of gelling agents act primarily by thickening the non-aqueous portion of the composition:

abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, itriisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

Exemplary gelling agents useful in the present invention include, but are not limited to,

| | |
|---|---|
| Polyethylene Glycol &Propylene Glycol & | (ACULYN 44) |
| Water | |
| Ammonium Acrylatedimethyltaurate/VP | (ARISTOFLEX AVC) |
| Copolymer | |
| Glyceryl Stearate &PEG 100 Stearate | (ARLACEL 165) |
| Polyethylene(2)Stearyl Ether | (BRIJ 72) |
| Polyoxyethylene(21)Stearyl Ether | (BRIJ 721) |
| Silica | (CAB-O-SIL) |
| Polyquaternium 10 | (CELQUAT CS230M) |
| Cetyl Alcohol | |
| Cetearyl Alcohol &Cetereth 20 | (COSMOWAX P) |
| Cetearyl Alcohol &Dicetyl Phosphate & | (CRODAFOS CES) |
| Ceteth-10 Phosphate | |
| Ceteth-20 Phosphate &Cetearyl Alcohol & | (CRODAFOS CS-20 |
| Dicetyl Phosphate | Acid) |
| Cetearyl Alcohol &Cetereth 20 | (EMULGADE NI 1000) |
| Sodium Magnesium Silicate | (LAPONITE XLG) |
| Cetyl Alcohol &Stearyl Alcohol & | (MACKADET CBC) |
| Stearalkonium Chloride &Dimethyl | |
| Stearamine &Lactic Acid | |
| Cetearyl Alcohol & | (MACKERNIUM |
| Stearamidopropyldimethylamine & | Essential) |
| Stearamidopropylalkonium Chloride | |
| Stearalkonium Chloride | (MACKERNIUM SDC- |
| | 85) |
| Cetearyl Alcohol & | (MACKERNIUM Ultra) |
| Stearamidopropyldimethylamine & | |
| Stearamidopropylalkonium | |
| Chloride &Silicone Quaternium 16 | |
| Cetearyl Alcohol &Cetearyl Glucoside | (MONTANOV 68EC) |
| Hydroxyethylcellulose | (NATROSOL 250 HHR |
| | CS) |
| Polyquaternium-37 &Mineral Oil & | (SALCARE SC 95) |
| Trideceth-6 | |
| Polyquaternium-32 &Mineral Oil & | (SALCARE SC 96) |
| Trideceth-6 | |
| Stearic Acid | |
| Cetyl Hydroxyethylcellulose | (NATROSOL Plus 330 |
| | CS) |
| Polyvinyl Alcohol, PVP-K30, Propylene | |
| Glycol | |
| Stearic Acid, Behenyl Alcohol, Glyceryl | (PROLIPID 141) |
| Stearate, Lecithin, C12-16 Alcohols, | |
| Palmic Acid | |
| Beeswax | (saponified |
| | beeswax) |
| Beeswax | (synthetic |
| | beeswax) |
| Water, Beeswax, Sesame Oil, Lecithin, | (beesmilk) |
| Methyl paraben | |
| Polyquaternium 10 | (CELQUAT SC240C) |
| Sodium Acrylate/Sodium Acrylodimethyl | (SIMULGEL EG) |
| Taurate Copolymer &Isohexadecane & | |
| Polysorbate 80 | |
| Polyquaternium 44 | (LUVIQUAT Care) |

To demonstrate the new and unexpected results provided by a method of the present invention, the following compositions were prepared and the ability of the method to control Gram positive and Gram negative bacteria, and to control rhinovirus, was determined. The weight percentage listed in each of the following compositions represents the actual, or active, weight amount of each ingredient present in a composition used in the present method of lowering skin pH. The compositions were prepared by blending the ingredients, as understood by those skilled in the art and as described below.

The following methods are used in the preparation and testing of the compositions:

a) Determination of Rapid Germicidal (Time Kill) Activity of Antibacterial Products. The activity of antibacterial compositions is measured by the time kill method, whereby the survival of challenged organisms exposed to an antibacterial test composition is determined as a function of time. In this test, a diluted aliquot of the composition is brought into contact with a known population of test bacteria for a specified time period at a specified temperature. The test composition is neutralized at the end of the time period, which arrests the antibacterial activity of the composition. The percent or, alternatively, log reduction from the original bacteria population is calculated.

In general, the time kill method is known to those skilled in the art.

The composition can be tested at any concentration up to 100%. The choice of which concentration to use is at the discretion of the investigator, and suitable concentrations are readily determined by those are tested at 50% dilution, whereas nonviscous samples are not diluted. The test sample is placed in a sterile 250 ml beaker equipped with a magnetic stirring bar and the sample volume is brought to 100 ml, if needed, with sterile deionized water. All testing is performed in triplicate, the results are combined, and the average log reduction is reported.

The choice of contact time period also is at the discretion of the investigator. Any contact time period can be chosen. Typical contact times range from 15 seconds to 5 minutes, with 30 seconds and 1 minute being typical contact times. The contact temperature also can be any temperature, typically room temperature, or about 25 degrees Celsius.

The bacterial suspension, or test inoculum, is prepared by growing a bacterial culture on any appropriate solid media (e.g., agar). The bacterial population then is washed from the agar with sterile physiological saline and the population of the bacterial suspension is adjusted to about 10⁸ colony forming units per ml (cfu/ml).

The table below lists the test bacterial cultures used in the tests and includes the name of the bacteria, the ATCC (American Type Culture Collection) identification number, and the abbreviation for the name of the organism used hereafter. S. aureus is a Gram positive bacteria, whereas E. coli, K. pneum, and S. choler. are Gram negative bacteria.

| Organism Name | ATCC # | Abbreviation |
|---|---|---|
| Staphylococcus aureus | 6538 | S. aureus |
| Escherichia coli | 11229 | E. coli |
| Klebsiellia pneumoniae | 10031 | K. pneum. |
| Salmonella choleraesuis | 10708 | S. choler. |

The beaker containing the test composition is placed in a water bath (if constant temperature is desired), or placed on a magnetic stirrer (if ambient laboratory temperature is desired). The sample then is inoculated with 1.0 ml of the test bacteria suspension. The inoculum is stirred with the test composition for the predetermined contact time. When the contact time expires, 1.0 ml of the test composition/bacteria mixture is transferred into 9.0 ml of Neutralizer Solution. Decimal dilutions to a countable range then are made. The dilutions can differ for different organisms. Selected dilutions are plated in triplicate on TSA+ plates (TSA+ is Trypticase Soy Agar with Lecithin and Polysorbate 80). The plates then are incubated for 24±two hours, and the colonies are counted for the number of survivors and the calculation of percent or log reduction. The control count (numbers control) is determined by conducting the procedure as described above with the exception that deionized water is used in place of the test composition. The plate counts are converted to cfu/ml for the numbers control and samples, respectively, by standard microbiological methods.

The log reduction is calculated using the formula

Log reduction=log₁₀(numbers controlled)−log₁₀(test sample survivors).

The following table correlates percent reduction in bacteria population to log reduction:

| % Reduction | Log Reduction |
|---|---|
| 90 | 1 |
| 99 | 2 |
| 99.9 | 3 |
| 99.99 | 4 |
| 99.999 | 5 |

b) Antiviral Residual Efficacy Test

References: S. A. Sattar, Standard Test Method for Determining the Virus-Eliminating Effectiveness of Liquid Hygienic Handwash Agents Using the Fingerpads of Adult Volunteers, Annual Book of ASTM Standards. Designation E1838-96, incorporated herein by reference in its entirety, and referred to as “Sattar I”; and S. A. Sattar et al., Chemical Disinfection to Interrupt Transfer of Rhinovirus Type 14 from Environmental Surfaces to Hands, Applied and Environmental Microbiology, Vol. 59, No. 5, May, 1993, pp. 1579-1585, incorporated herein by reference in its entirety, and referred to as “Sattar II.”

The method used to determine the Antiviral Index of the present invention is a modification of that described in Sattar I, a test for the virucidal activity of liquid hand washes (rinse-off products). The method is modified in this case to provide reliable data for leave-on products.

The modifications from Sattar I include the product being delivered directly to skin as described below, virus inoculation of the fingerpads as described below, and viral recovery using ten-cycle washing. The inoculated skin site then is completely decontaminated by treating the area with 70% dilution of ethanol in water.

Procedure:

Ten-Minute Test:

Subjects (5 per test product) initially wash their hands with a nonmedicated soap, rinse the hands, and allow the hands to dry.

The hands then are treated with 70% ethanol and air dried.

Test product (1.0 ml) is applied to the hands, except for the thumbs, and allowed to dry.

About 10 minutes (±30 seconds) after product application, 10 μl of a Rhinovirus 14 suspension (ATCC VR-284, approximately 1×10⁶ PFU (plaque-forming units)/ml) is topically applied using a micropipette to various sites on the hand within a designated skin surface area known as fingerpads. At this time, a solution of rhinovirus also is applied to the untreated thumb in a similar manner.

After a dry-down period of 7-10 minutes, the virus then is eluted from each of the various skin sites with 1 ml of eluent (Earle's Balanced Salt Solution (EBSS) with 25% Fetal Bovine Serum (FBS)+1% pen-strep-glutamate), washing 10 times per site.

The inoculated skin site then is completely decontaminated by rinsing the area with 70% ethanol. Viral titers are determined using standard techniques, i.e., plaque assays or TCID₅₀ (Tissue Culture Infectious Dose).

One-Hour Test:

Subjects are allowed to resume normal activities (with the exception of washing their hands) between the 1-hour and 3-hour timepoints. After one hour, a rhinovirus suspension is applied to and eluted from designated sites on the fingerpads exactly as described in above for the 10-minute test.

### Example 1

A composition capable of lowering skin pH in accordance with the present invention was prepared by admixing the following ingredients at the indicated weight percentages until homogeneous.

| Ingredient | Weight Percent |
|---|---|
| Citric acid | 2.1 |
| Water | q.s. |

The composition is applied to the skin of an individual in a quantity sufficient to create a surface concentration of at least about 10 micrograms of citric acid per square centimeter of skin surface. The skin pH is reduced from an ambient value of about 5 to 5.5 to an initial value after application of the composition of about 2 to 2.5. The skin is maintained at a pH of less than 3.5 for up to about five hours after application. The skin exhibits an excellent control of viruses and bacteria.

### Example 2

This example demonstrates the surprising and unexpected relationship between skin pH and antirhinoviral efficacy. While prior acidic compositions were applied to the skin of the user to provide antiviral, and particularly antirhinoviral, properties, it has been found that simply lowering the skin pH is not sufficient to assure antiviral efficacy. More specifically, to achieve a highly efficacious antiviral efficacy over an extended period of time, such as four hours, the pH of the skin must be maintained at less than 4 for the entire four hours.

In this example, antirhinoviral activity is assessed 5 minutes after application of an organic acid solution having a pH adjusted over a range of pH values in order to determine the effective pH limits of the compositions. Test solutions containing 1% citric acid and 1% malic acid, each by weight, in aqueous 10% ethanol solvent were prepared. The pH values of the solutions were adjusted by the addition of triethanolamine to provide compositions having a pH value indicated below:

| Composition | pH |
|---|---|
| 2A | 2.3 |
| 2B | 4.5 |
| 2C | 5.6 |

The antirhinoviral efficacy of each solution was measured using the in vivo antirhinoviral fingerpad test procedure. The following table lists the composition tested, the skin pH after application of the test solution, the average log₁₀ (viral titer inoculum applied to the fingers of volunteers), and the average log₁₀ (viral titer recovered from the fingers). The test solution was applied to all fingers of the volunteers except the thumbs. The fingers then were allowed to dry for 5 minutes, and the rhinovirus inoculum was applied to all fingers. The thumbs serve as a negative control, and the inoculum was determined by the rhinovirus titer recovered from the thumbs. In this test, two volunteers were used for each pH tested. The skin pH reported is the average for the two volunteers.

| | | | log₁₀ (Virus | log₁₀ (Virus |
|---|---|---|---|---|
| Composition | Composition pH | Skin pH | inoculum) | recovered) |
| 2A | 2.3 | 3.0 | 3.9 | 0.23 |
| 2B | 4.5 | 4.7 | 4.0 | 3.1 |
| 2C | 5.6 | 5.6 | 4.1 | 3.6 |

This example clearly shows that a skin pH value of 5.6 or 4.7 is ineffective at eliminating rhinovirus, whereas a skin pH of 3.0 is highly effective at eliminating or essentially eliminating rhinovirus from human skin. An average log recovery of less than 1 indicates fewer than a virus particle remaining on average after the test, which also means that the virus level was below the limit of detection in the test.

### Example 3

The following antirhinoviral composition, which capable of reducing skin pH, was prepared and applied to the fingerpads of human volunteers:

Composition 2D

| Material | Percent (by weight) |
|---|---|
| Ethanol | 70.0 |
| Deionized water | 19.8 |
| ULTREZ ® 20¹⁾ | 1.0 |
| Isopropyl Palmitate | 1.0 |
| Mineral oil | 1.0 |
| DC 200 silicone fluid | 1.0 |
| Cetyl alcohol | 1.0 |
| Citric acid | 2.0 |
| Malic acid | 2.0 |
| GERMABEN II²⁾ | 1.0 |
| Triethanolamine | 0.05 |
| | 100.0 |

¹⁾Acrylate/C10-30 Alkyl Acrylate Crosspolymer;
²⁾Preservative containing propylene glycol, diazolidinyl urea, methylparaben, and propylparaben.

### The pH of Sample 2 was 3.1.

In the test, composition 2D was applied to the fingerpads of all fingers, except the thumbs, of eight volunteers. The thumbs were control sites. The volunteers were divided into fours groups of two each. Each group I-IV then was challenged at a predetermined time with rhinovirus titer on all the fingerpads of each hand to determine the time-dependent efficacy of the test composition. At the time appropriate for each group, the skin pH of the fingerpads also was measured to determine the time course of skin pH in response to the test composition. The predetermined test time for rhinoviral challenge and skin pH measurement for each group I-IV were 5 minutes, 1 hour, two hours, and four hours, respectively. The following table shows the average log (rhinoviral titer inoculum), average skin pH, and average log (rhinoviral titer recovered) from the test fingerpads of the volunteers in the study, organized by group.

| | Initial skin | | | |
|---|---|---|---|---|
| | pH after | Skin pH at | Log [Inoculum | Log [Recovered |
| | application | test time | Titer] | Titer] |
| Group | (average) | (average) | (average) | (average) |
| I | 3.0 | 3.0 | 3.9 | 0.23 |
| II | 2.8 | 3.4 | 4.0 | 0.23 |
| III | 3.0 | 3.8 | 3.8 | 0.23 |
| IV | 3.0 | 3.8 | 4.3 | 0.23 |

The data for each group (i.e., different time points) shows that the average recovered rhinoviral titer is less than 1 virus particle, or below the detection limit of the test. This data illustrates the efficacy of the present method after four hours and further demonstrates that a pH of less than about 4 is effective at completely eliminating a virus challenge.

### Example 4

The clean fingerpads of test subjects were treated with the following compositions. Baseline skin pH readings were measured from the fingerpads prior to treatment with the compositions. Skin pH measurements also were taken immediately after the composition dried on the fingerpads, then again after four hours.

| | | | | | % |
|---|---|---|---|---|---|
| | | Average | Average | Viral | Hands |
| | | Skin pH | Skin pH | Log 10 | with |
| Sample | Composition (by wt %) | (T = 0) | (T = 4 hr) | Reduction | Virus |
| A | 2% citric acid, 2% | 2.81 | 3.23 | >3 log₁₀ | 0 |
| | malic acid, 62% ETOH, | | | | |
| | 1.25% hydroxyethyl- | | | | |
| | cellulose | | | | |
| B | 2% citric acid, | 2.64 | 3.03 | >3 log₁₀ | 0 |
| | 2% tartaric acid, | | | | |
| | 62% ETOH, 1.25% | | | | |
| | hydroxyethylcellulose | | | | |
| C | 2% malic acid, 2% | 2.66 | 2.94 | >3 log₁₀ | 0 |
| | tartaric acid, 62% | | | | |
| | ETOH, 1.25% hydroxy- | | | | |
| | ethylcellulose | | | | |
| D | 62% ETOH, 1.25% hydroxyethylcellulose | 5.53 | 5.13 | <0.5 log₁₀ | 100 |
| E | 2% citric acid, 2% | 2.90 | 3.72 | >3 log₁₀ | 0 |
| | malic acid, 70% ETOH, | | | | |
| | 1% polyacrylic acid | | | | |
| F | 70% ETOH, 1% poly- | 4.80 | 5.16 | 2.0 log₁₀ | 100 |
| | acrylic acid | | | | |
| G | 70% ETOH, 1.25% hydroxyethylcellulose | 5.3 | 5.25 | <0.5 log₁₀ | 100 |

¹⁾ETOH is ethanol

Four hours after treatment of the fingerpads with Samples A-G, Rhinovirus 39 at a titer of 1.3×10³ pfu (plaque forming units) was applied to fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 39, i.e., a greater than 3 log reduction, was achieved using the acid-containing compositions containing a mixture of two of citric acid, malic acid, and tartaric acid.

### Example 5

### Antibacterial Activity

Log Reduction

| | S. aureus | | E. coli | |
|---|---|---|---|---|
| | ATCC 6538 | | ATCC 11229 | |
| Sample | 30 seconds¹⁰ | 60 seconds¹⁾ | 30 seconds | 60 seconds |
| A | >4.91 | >4.91 | >5.00 | >5.00 |
| B | >4.91 | >4.91 | >5.00 | >5.00 |

¹⁾Contact time on the skin
A. 62% Ethanol, 2% citric acid, 2% malic acid, 1.25% hydroxyethylcellulose
B. 62% Ethanol, 2% citric acid, 2% malic acid, 1.25% hydroxyethylcellulose, and skin emollients

This example illustrates that compositions of the present invention also provide a rapid and broad spectrum antibacterial activity.

### Example 6

The clean fingerpads of test subjects were treated with the following composition. Baseline skin pH readings were measured from the fingerpads prior to treatment with the compositions. Skin pH measurements also were taken immediately after the composition dried on the fingerpads.

Immediately after treatment of the fingerpads with the composition, Rhinovirus 14 at a titer of 1.4×10⁴ pfu (plaque forming units) was applied to the fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 14 was achieved with the acid-containing composition resulting in a 4 log reduction.

| | | | Viral Log 10 | % |
|---|---|---|---|---|
| | | Solution | Reduction 30 | Hands with |
| Sample | Composition (by wt %) | pH | seconds | Virus |
| A | 2% citric acid, 2% | 3.10 | 4 log | 0 |
| | malic acid, 70% | | | |
| | ETOH, 1% polyacrylic | | | |
| | acid | | | |

### Example 7

The following compositions were prepared to test the effect of organic acids and organic acid blends on skin pH and antiviral efficacy.

| | | Average | Average | Viral |
|---|---|---|---|---|
| | | Skin pH | Skin pH | Log10 |
| Sample | Composition (by wt %) | (T = 0) | (T = 2 hr) | Reduction |
| A | 4% citric acid in 70% | 2.97 | 3.64 | >3 log₁₀ |
| | ethanol/water | | | |
| B | 4% malic acid in 70% | 2.91 | 3.94 | >3 log₁₀ |
| | ethanol/water | | | |
| C | 2% citric acid and 2% | 2.99 | 3.38 | >3 log₁₀ |
| | malic acid in 70% | | | |
| | ethanol/water | | | |
| D | 4% tartaric acid in 70% | 2.56 | 3.0 | >3 log₁₀ |
| | ethanol/water | | | |

The clean fingerpads of the test subjects were treated with Samples A-D. Baseline skin pH readings were measured from the fingerpads prior to treatment with a composition. Skin pH measurements also were taken immediately after the composition dried on the fingerpads, and again after two hours.

All Samples A-D suppressed skin pH to below 4 for two hours. The combination of citric acid and malic acid (Sample C) maintained a lower pH at two hours than the same acids used singly 4 Samples A and B). The 4% tartaric acid composition (Sample D) showed the greatest suppression of skin pH.

Two hours after treatment of the fingerpads with the solutions, Rhinovirus 39 at a titer of 4×10⁴ pfu was applied to fingerpads. The virus was dried on the fingerpads for 10 minutes, then the fingerpads were rinsed with a viral recovery broth containing 75% EBSS and 25% FBS with 1× antibiotics. The sample was serially diluted in viral recovery broth and plated onto H1-HeLa cells. Titers were assayed as per the plaque assay. Complete inactivation of Rhinovirus 39 was achieved resulting in a greater than 3 log reduction.

The following examples illustrate that polymeric acids, and especially an acrylic acid homopolymer or copolymer, in the presence of alcohol impart antiviral efficacy. The polymeric acids have a low pH and good substantivity to skin, which effectively maintains a low skin pH over time, and helps provide a persistent antiviral efficacy.

A synergistic effect on the lowering of skin pH was demonstrated with using acrylic acid-based polymer in the presence of alcohol. However, an acrylic acid-based polymer in the absence of an alcohol did not maintain a reduced skin pH to the same degree over time. Importantly, skin pH reduction is less dependent on composition pH when a polymeric acid is used in conjunction with an alcohol. The synergy demonstrated between the polymeric acid and the alcohol was unexpected and is a novel way of providing the lowered skin pH that provides a desired antiviral efficacy.

A synergistic effect on a rapid and persistent antiviral activity also is demonstrated when an acrylic acid-based polymer is used in conjunction with polycarboxylic acids. It has been found that utilizing a low amount of a polymeric acid (e.g., about 0.1% to about 2%, by weight) together with a polycarboxylic acid, like citric acid, malic acid, tartaric acid, and mixtures thereof, enhances the antiviral activities of the polycarboxylic acids. This synergistic effect allows a reduction in the polycarboxylic acid concentration in an antiviral composition, without a concomitant decrease in antiviral efficacy. This reduction in polycarboxylic acid concentration improves composition mildness by reducing the irritation potential of the composition.

### Example 8

The following compositions were prepared to examine the effectiveness of polycarboxylic acid blends and a single polycarboxylic acid composition, each in combination with polyacrylic acid and ethanol, on antiviral efficacy. A preferred antiviral composition contains the least amount of organic acid required to demonstrate a persistent antiviral efficacy.

The compositions were applied to the fingerpads of clean hands. After the indicated times, about 10³ to 10⁴ pfu of Rhinovirus 39 was applied to the hands and allowed to dry for 10 minutes. The virus was recovered by rinsing the hands with viral recovery broth. The samples then were diluted serially in viral recovery broth and plated on H1-HeLa cells. Viral titers were determined by plaque assay. The percentage of hands that were positive for rhinovirus is summarized below.

| | | | % of |
|---|---|---|---|
| | | | Hands Positive |
| Composition (by wt %) | Time | | for Rhinovirus |
| 70% ethanol | 15 | min. | 100% |
| 1% citric acid/1% malic acid/10% | 1 | hr. | 100% |
| ethanol/water | | | |
| 1% polyacrylic acid/4% citric acid/70% | 4 | hrs. | 91% |
| ethanol/water | | | |
| 1% polyacrylic acid/1% citric acid/1% | 4 | hrs. | 0% |
| malic acid/70% ethanol/water | | | |

A composition containing 70% ethanol alone was (1%) and malic acid (1%) lost effectiveness against rhinovirus after one hour because 100% of the hands were found to be positive for rhinovirus. In contrast, when a composition containing 1% citric and 1% malic acids are applied to the hands in combination with polyacrylic acid and 70% ethanol, no virus was detected on the hands after four hours. A single acid (4% citric acid) in combination with a polyacrylic acid and ethanol was less effective against rhinovirus because 91% of hands were found to be positive for rhinovirus after four hours.

This data demonstrates that using a polyacrylic acid and ethanol allows the use of a lower concentration of polycarboxylic acid to achieve a desired antiviral efficacy.

### Example 9

The use of a polyacrylic acid and ethanol in a composition suppresses skin pH to a value below the solution pH, as demonstrated in Example 7. To test whether antiviral compositions containing citric acid, malic acid, polyacrylic acid, and ethanol can be buffered to a higher solution pH and still provide a skin pH at or below pH 4 to obtain a persistent antiviral activity, the following compositions were prepared.

| | | | Skin | Skin | |
|---|---|---|---|---|---|
| | | Solution | pH | pH | Viral |
| Sample | Composition (by wt %) | pH | Initial | 4 hrs. | Reduction |
| A | 1% ULTREZ 20/2% citric | 3.2 | 2.9 | 3.7 | >3 log₁₀ |
| | acid/2% malic acid/70% | | | | |
| | ethanol | | | | |
| B | 1% ULTREZ 20/2% citric | 4.34 | 3.4 | 3.7 | >3 log₁₀ |
| | acid/2% malic acid/70% | | | | |
| | ethanol | | | | |
| C | 1% ULTREZ 20/2% citric | 4.65 | 3.6 | 3.8 | >3 log₁₀ |
| | acid/2% malic acid/70% | | | | |
| | ethanol | | | | |

The compositions (1.8 mL) were applied to the thumb, index, and middle fingers of clean hands. Skin pH readings were measured prior to treatment (baseline), immediately after the fingers were dry, and again after four hours. The average of the skin pH values are plotted above.

Initial skin pH of skin treated with Samples A-C were suppressed to between pH 2.9 and 3.6, wherein the lower the solution pH, the lower the initial skin pH. However, after four hours, the skin pH for all three compositions was about pH 3.7. Consistent with previous examples, solution pH did not predict subsequent skin pH.

The viral efficacy of Samples A-C against Rhinovirus 39 also was tested. A viral load of about 10³ pfu was spread over the thumb, index, and middle fingers of each treated hand and allowed to dry for 10 minutes. The fingers then were rinsed with viral recovery broth and samples were diluted serially and plated on H1-HeLa cells. Viral titers were measured using the plaque assay. No virus was recovered from any of the hands indicating that all three Samples A-C have antiviral efficacy.

This data demonstrates than when citric acid and malic acid are utilized in a composition in combination with a polyacrylic acid and ethanol, the pH of the solution can be buffered to a higher, e.g., milder and safer, pH for application to the skin, while still retaining an ability to suppress skin pH and exhibit antiviral activity.

The method of the present invention can be practiced using, for example, hand cleansers, surgical scrubs, body splashes, antiseptics, disinfectants, hand sanitizer gels, deodorants, and similar personal care products. Additional types of compositions that can be used in the present method include foamed compositions, such as creams, mousses, and the like, and compositions containing organic and inorganic filler materials, such as emulsions, lotions, creams, pastes, and the like. The method further can be practiced on hard surfaces, for example, sinks and countertops in hospitals, food service areas, and meat processing plants.

The method also can be practiced by incorporating a suitable compound or composition into a web material to provide a wiping article. The wiping article can be used to control microbes on animate or inanimate surfaces.

In one embodiment of the present invention, a person suffering from a rhinovirus cold, or who is likely to be exposed to other individuals suffering from rhinovirus colds, can apply a compound or composition capable of lowering skin pH to less than 4 to his or her hands. This application kills bacteria and inactivates rhinovirus particles present on the hands. The applied compound or composition, either rinsed off or allowed to remain on the hands, provides a persistent antiviral activity. Rhinovirus particles therefore are not transmitted to noninfected individuals via hand-to-hand transmission. The amount of the compound or composition applied, the frequency of application, and the period of use will vary depending upon the level of disinfection desired, e.g., the degree of microbial contamination.

The present method provides the advantages of a broad spectrum kill of Gram positive and Gram negative bacteria, and a viral control, in short contact times. The short contact time for a substantial log reduction of bacteria is important in view of the typical 15 to 60 second time frame used to cleanse and sanitize the skin and inanimate surfaces. The method also imparts a persistent antiviral activity to the contacted surface.

Obviously, many modifications and variations of the invention as hereinbefore set forth can be made without departing from the spirit and scope thereof, and, therefore, only such limitations should be imposed as are indicated by the appended claims.

What is claimed is:

1. A method of controlling viruses and bacteria on mammalian skin comprising contacting the skin with a compound or a composition capable of lowering skin pH to less than about 4 for at least about 0.5 hours.

2. The method of claim 1 wherein the compound or the composition lowers skin pH to less than about 4 for at least about two hours.

3. The method of claim 1 wherein the compound or the composition lowers skin pH to less than about 4 for up to about eight hours.

4. The method of claim 1 wherein the compound or the composition is capable of lowering skin pH to less than about 3.5.

5. The method of claim 1 wherein the compound or the composition is capable of lowering skin pH to less than about 3.0.

6. The method of claim 1 wherein the compound or the composition is allowed to remain on the skin.

7. The method of claim 1 wherein the compound or the composition is rinsed from the skin.

8. The method of claim 1 wherein the compound capable of lowering skin pH is selected from the group consisting of (a) an organic acid, (b) an inorganic acid, (c) an inorganic salt comprising a cation having a valence of 2, 3, or 4 and a counterion capable of lowering the skin pH to less than about 4, (d) an aluminum, zirconium, or aluminum-zirconium complex, and (e) mixtures thereof.

9. The method of claim 1 wherein the compound capable of lowering skin pH is present in a composition in an amount of about 0.05% to about 6%, by weight of the composition.

10. The method of claim 1 wherein the compound capable of lowering skin pH is applied to the skin in an amount of at least 10 micrograms of the compound per square centimeter of skin surface.

11. The method of claim 8 wherein the compound comprises an organic acid having a water solubility of at least about 0.05% by weight, at 25° C.

12. The method of claim 8 wherein the organic acid comprises a monocarboxylic acid, a polycarboxylic acid, a polymeric acid having a plurality of carboxylic, phosphate, sulfonate, and/or sulfate moieties, anhydrides thereof, or mixtures thereof.

13. The method of claim 12 wherein the a monocarboxylic acid has a structure RCO₂H, wherein R is C₁₋₃alkyl, hydroxyC₁₋₃alkyl, haloC₁₋₃alkyl, phenyl, or substituted phenyl.

14. The method of claim 13 wherein the monocarboxylic acid is selected from the group consisting of acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof.

15. The method of claim 12 wherein the polycarboxylic acid contains two to four carboxylic acid groups, and optionally one or more hydroxyl group, amino group, or both.

16. The method of claim 15 wherein the polycarboxylic acid is selected from the group consisting of malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

17. The method of claim 15 wherein the polycarboxylic acid comprises an anhydride of the polycarboxylic acid.

18. The method of claim 12 wherein the polymeric acid has a molecular weight of about 500 to about 10,000,000 g/mol.

19. The method of claim 12 wherein the polymeric acid has a T_{g} of less than about 250-C.

20. The method of claim 12 wherein the polymeric acid is capable of forming a substantive film on the skin.

21. The method of claim 18 wherein the polymeric acid is water soluble or water dispersible.

22. The method of claim 12 wherein the polymeric acid is selected from the group consisting of a polymeric carboxylic acid, a polymeric sulfonic acid, a sulfated polymer, a polymeric phosphoric acid, and mixtures thereof.

23. The method of claim 12 wherein the polymeric acid comprises a homopolymer or a copolymer of acrylic acid.

24. The method of claim 8 wherein the organic acid comprises a polycarboxylic acid and a polymeric carboxylic acid.

25. The method of claim 24 wherein the polycarboxylic acid comprises citric acid, malic acid, tartaric acid, or mixtures thereof, and the polymeric carboxylic acid comprises a homopolymer or a copolymer of acrylic acid or methacrylic acid.

26. The method of claim 25 wherein the polymeric acid comprises a homopolymer or a copolymer of acrylic acid.

27. The method of claim 8 wherein the inorganic acid is selected from the group consisting of phosphorous acid, phosphoric acid, pyrophosphoric acid, polyphosphoric acid, and mixtures thereof.

28. The method of claim 8 wherein the inorganic salt comprises a cation selected from the group consisting of magnesium, calcium, barium, aluminum, iron, cobalt, nickel, copper, zinc, zirconium, and tin.

29. The method of claim 28 wherein the counterion is selected from the group consisting of bisulfate, sulfate, dihydrogen phosphate, monohydrogen phosphate, chloride, iodide, bromide, and nitrate.

30. The method of claim 29 wherein the counterion of the inorganic salt comprises a chloride.

31. The method of claim 8 wherein the inorganic salt comprises a divalent zinc salt.

32. The method of claim 8 wherein the aluminum, zirconium, or aluminum-zirconium complex comprises an aluminum complex.

33. The method of claim 1 wherein the composition further comprises 0.1% to about 5% of an antimicrobial agent is selected from the group consisting of a phenolic antibacterial agent, a quaternary ammonium antimicrobial agent, an anilide, a bisguanidine, and mixtures thereof.

34. The method of claim 33 wherein the antimicrobial agent comprises a phenolic antimicrobial agent selected from the group consisting of:
(a) a 2-hydroxydiphenyl compound having the structure 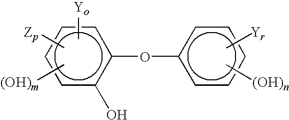
wherein Y is chlorine or bromine, Z is SO₃H, NO₂, or C₁-C₄ alkyl, r is 0 to 3, o is 0 to 3, p is 0 or 1, m is 0 or 1, and n is 0 or 1;
(b) a phenol derivative having the structure 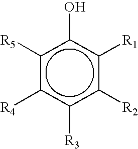
wherein R₁ is hydro, hydroxy, C₂-C₄ alkyl, chloro, nitro, phenyl, or benzyl, R₂ is hydro, hydroxy, C₁-C₆ alkyl, or halo, R₃ is hydro, C₁-C₆ alkyl, hydroxy, chloro, nitro, or a sulfur in the form of an alkali metal salt or ammonium salt, R₄ is hydro or methyl, and R₅ is hydro or nitro;
(c) a diphenyl compound having the structure 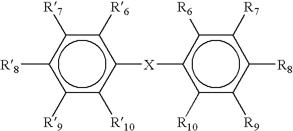
wherein X is sulfur or a methylene group, R₆ and R′₆ are hydroxy, and R₇, R′₇, R₈, R′₈, R₉, R′₉, R¹⁰, and R′₁₀, independent of one another, are hydro or halo; and
(d) mixtures thereof.

35. The method of claim 33 wherein the antimicrobial agent comprises a quaternary ammonium antimicrobial agent having a structure: 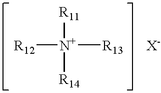
wherein R₁₁ is an alkyl, aryl, or alkaryl substituent containing 6 to 26 carbon atoms, R₁₂, R₁₃, and R₁₄, independently, are substituents containing no more than twelve carbon atoms, and X is an anion selected from the group consisting of halo, methosulfate, ethosulfate, and p-toluenesulfonyl, or 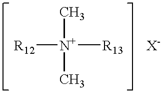
wherein R₁₂ and R₁₃, independently, are C₈-C₁₂alkyl, or R₁₂ is C₁₂-C₁₆alkyl, C₈-C₁₈alkylethoxy, or C₈-C₁₆alkylphenylethoxy, and R₁₃ is benzyl, and X is halo, methosulfate, ethosulfate, or p-toluenesulfonate.

36. The method of claim 33 wherein the antimicrobial agent comprises an anilide or a bisguanidine selected from the group consisting of triclocarban, carbanilide, salicylanilide, tribromosalan, tetrachlorosalicylanilide, fluorosalan, chlorhexidine gluconate, chlorhexidine hydrochloride, and mixtures thereof.

37. The method of claim 1 wherein the composition further comprises a disinfecting alcohol in an amount of 10% to about 90%, by weight, of the composition.

38. The method of claim 37 wherein the disinfecting alcohol comprises one or more C₁₋₆ alcohol.

39. The method of claim 37 wherein the disinfecting alcohol is selected from the group consisting of methanol, ethanol, isopropyl alcohol, n-butanol, n-propyl alcohol, and mixtures thereof.

40. The method of claim 1 wherein the composition further comprises up to about 30%, by weight, of a polyhydric solvent selected from the group consisting of a diol, a triol, and mixtures thereof.

41. The method of claim 1 wherein the composition further comprises up to about 30%, by weight, of a hydrotrope.

42. The method of claim 1 wherein the composition further comprises 0.1% to about 5%, by weight, of a gelling agent.

43. The method of claim 42 wherein the gelling agent comprises a natural gum, a synthetic polymer, a clay, an oil, a wax, or mixtures thereof.

44. The method of claim 1 wherein the composition further comprises 0.1% to about 5%, by weight, of a surfactant.

45. The method of claim 44 wherein the surfactant comprises an anionic, cationic, or ampholytic surfactant, or mixtures thereof.

46. The method of claim 1 wherein the skin has a log reduction against Gram positive bacteria of at least 2 after 30 seconds of contact, as measured against S. aureus.

47. The method of claim 1 wherein the skin has a log reduction against Gram negative bacteria of at least 2.5 after 30 seconds of contact, as measured against E. coli.

48. The method of claim 1 wherein the skin has a log reduction against an acid-labile virus of at least 4 after 30 seconds of contact.

49. The method of claim 1 wherein the skin has a log reduction against an acid-labile virus of at least 3 five hours after contact with the compound or composition.

50. The method of claim 1 wherein the skin has a log reduction against an acid-labile virus of at least 2 eight hours after contact with the compound or composition.

51. A method of reducing a bacteria and a virus population on a surface comprising contacting the surface with a compound or a composition capable of lowering the surface pH to less than 4 for 30 seconds to achieve a log reduction of at least 2 against S. aureus, a log reduction of at least 2.5 against E. coli, and a log reduction of at least 4 against an acid-labile virus.

52. The method of claim 51 wherein the acid-labile virus comprises a rhinovirus serotype.

53. The method of claim 51 further comprising a step of rinsing the composition from the surface.

54. The method of claim 51 wherein the surface is a skin of a mammal.

55. The method of claim 51 wherein the surface is a hard, inanimate surface.

56. The method of claim 51 wherein the surface has a persistent antiviral activity for a period of up to about six hours.

57. A method of inactivating viruses on a surface for up to about eight hours comprising a step of topically applying a compound or a composition capable of lowering surface pH to less than 4 to a surface in need of such treatment.

58. The method of claim 57 wherein the viruses are inactivated for up to about six hours.

59. The method of claim 57 wherein the surface is animate.

60. The method of claim 57 wherein the surface is inanimate.

61. The method of claim 57 wherein rhinoviruses, picornaviruses, adenoviruses, and rotaviruses are inactivated.

62. The method of claim 57 wherein acid-labile rinses are inactivated.

63. The method of claim 57 wherein picornaviruses are inactivated.

64. The method of claim 57 wherein rhinoviruses are inactivated.

65. A method of improving the overall health of a mammal by reducing exposure to viruses and bacteria comprising the steps of:
(a) topically applying a compound or a composition capable of lowering a surface pH to less than 4 to a surface of the mammal that is prone to viral and/or bacterial contamination; and
(b) allowing the surface to dry.

66. A method of protecting an individual against infection by rhinoviruses comprising a step of applying a compound or a composition capable of lowering skin pH to less than 4 to hands of the individual in an amount sufficient to eradicate rhinoviruses.

67. The method of claim 66 wherein the compound or the composition is applied prior to the individual being exposed to rhinoviruses.

68. The method of claim 66 wherein the compound or the composition is applied multiple times within a twenty-four hour period.

69. The method of claim 66 wherein the compound or the composition is rinsed from the hands.

70. The method of claim 66 wherein the compound or the composition is allowed to dry and remain on the hands.
